SEQUENCE LISTING (36 sequences)

SEQ ID NO: 1 - protein, 28 aa, Artificial Sequence
  Description of Artificial Sequence Synthetic peptide
GGCRGDMFGCGGKKKKKKKKKKKKKKKK

SEQ ID NO: 2 - protein, 27 aa, Artificial Sequence
  Description of Artificial Sequence Synthetic peptide
GGCRGDMFGCGKKKKKKKKKKKKKKKK

SEQ ID NO: 3 - protein, 26 aa, Artificial Sequence
  Description of Artificial Sequence Synthetic peptide
GGCRGDMFGCKKKKKKKKKKKKKKKK

SEQ ID NO: 4 - protein, 27 aa, Artificial Sequence
  Description of Artificial Sequence Synthetic peptide
KKKKKKKKKKKKKKKKGACRGDMFGCA

SEQ ID NO: 5 - protein, 8 aa, Artificial Sequence
  Description of Artificial Sequence Synthetic peptide
CRGDMFGC

SEQ ID NO: 6 - protein, 10 aa, Artificial Sequence
  Description of Artificial Sequence Synthetic peptide
GGCRGDMFGC

SEQ ID NO: 7 - protein, 11 aa, Artificial Sequence
  Description of Artificial Sequence Synthetic peptide
GGCRGDMFGCG

SEQ ID NO: 8 - protein, 11 aa, Artificial Sequence
  Description of Artificial Sequence Synthetic peptide
GGCRGDMFGCA

SEQ ID NO: 9 - protein, 11 aa, Artificial Sequence
  Description of Artificial Sequence Synthetic peptide
GACRGDMFGCA

SEQ ID NO: 10 - protein, 12 aa, Artificial Sequence
  Description of Artificial Sequence Synthetic peptide
GACDCRGDCFCA

SEQ ID NO: 11 - protein, 12 aa, Artificial Sequence
  Description of Artificial Sequence Synthetic peptide
GACRRETAWACA

SEQ ID NO: 12 - protein, 12 aa, Artificial Sequence
  Description of Artificial Sequence Synthetic peptide
GACRRETAWACG

SEQ ID NO: 13 - protein, 9 aa, Artificial Sequence
  Description of Artificial Sequence Synthetic peptide
CRRETAWAC

SEQ ID NO: 14 - protein, 5 aa, Artificial Sequence
  Description of Artificial Sequence Synthetic peptide
XSXGA

SEQ ID NO: 15 - protein, 12 aa, Artificial Sequence
  Description of Artificial Sequence Synthetic peptide
GACRGDMFGCGG

SEQ ID NO: 16 - protein, 12 aa, Artificial Sequence
  Description of Artificial Sequence Synthetic peptide
GAGPEILDVPST

SEQ ID NO: 17 - protein, 10 aa, Artificial Sequence
  Description of Artificial Sequence Synthetic peptide
GACQIDSPCA

SEQ ID NO: 18 - protein, 25 aa, Artificial Sequence
  Description of Artificial Sequence Synthetic peptide
GACRRETAWACGKGACRRETAWACG

SEQ ID NO: 19 - protein, 16 aa, Artificial Sequence
  Description of Artificial Sequence Synthetic peptide
KKKKKKKKKKKKKKKK

SEQ ID NO: 20 - protein, 27 aa, Artificial Sequence
  Description of Artificial Sequence Synthetic peptide
KKKKKKKKKKKKKKKKGACRGDMFGCA

SEQ ID NO: 21 - protein, 28 aa, Artificial Sequence
  Description of Artificial Sequence Synthetic peptide
KKKKKKKKKKKKKKKKGACRRETAWACG

SEQ ID NO: 22 - protein, 27 aa, Artificial Sequence
  Description of Artificial Sequence Synthetic peptide
KKKKKKKKKKKKKKKKGACRGDMFGCA

SEQ ID NO: 23 - protein, 28 aa, Artificial Sequence
  Description of Artificial Sequence Synthetic peptide
KKKKKKKKKKKKKKKKGACDCRGDCFCA

SEQ ID NO: 24 - protein, 27 aa, Artificial Sequence
  Description of Artificial Sequence Synthetic peptide
KKKKKKKKKKKKKKKKGGCRGDMFGCA

SEQ ID NO: 25 - protein, 26 aa, Artificial Sequence
  Description of Artificial Sequence Synthetic peptide
KKKKKKKKKKKKKKKKGACQIDSPCA

SEQ ID NO: 26 - protein, 31 aa, Artificial Sequence
  Description of Artificial Sequence Synthetic peptide
KKKKKKKKKKKKKKKKXSXGACRRETAWACG

SEQ ID NO: 27 - protein, 8 aa, Artificial Sequence
  Description of Artificial Sequence Synthetic peptide
CRGDMFGC

SEQ ID NO: 28 - protein, 9 aa, Artificial Sequence
  Description of Artificial Sequence Synthetic peptide
CRGDMFGCA

SEQ ID NO: 29 - protein, 10 aa, Artificial Sequence
  Description of Artificial Sequence Synthetic peptide
CDCRGDCFCA

SEQ ID NO: 30 - protein, 10 aa, Artificial Sequence
  Description of Artificial Sequence Synthetic peptide
CRRETAWACA

SEQ ID NO: 31 - protein, 10 aa, Artificial Sequence
  Description of Artificial Sequence Synthetic peptide
CRRETTAWAC

SEQ ID NO: 32 - protein, 10 aa, Artificial Sequence
  Description of Artificial Sequence Synthetic peptide
CRRETAWACG

SEQ ID NO: 33 - protein, 10 aa, Artificial Sequence
  Description of Artificial Sequence Synthetic peptide
CRGDMFGCGG

SEQ ID NO: 34 - protein, 10 aa, Artificial Sequence
  Description of Artificial Sequence Synthetic peptide
GPEILDVPST

SEQ ID NO: 35 - protein, 8 aa, Artificial Sequence
  Description of Artificial Sequence Synthetic peptide
CQIDSPCA

SEQ ID NO: 36 - protein, 23 aa, Artificial Sequence
  Description of Artificial Sequence Synthetic peptide
CRRETAWACGKGACRRETAWACG

(19) United States
(12) Patent Application Publication
(10) Pub. No.: US 2004/0014217 A1
(43) Pub. Date: Jan. 22, 2004

(54) Methods of transfection

(76) Inventor: Stephen Lewis Hart, London (GB)

Correspondence Address: NIXON & VANDERHYE, PC, 1100 N GLEBE ROAD, 8TH FLOOR, ARLINGTON, VA, 22201-4714, US

(21) Appl. No.: 10/296,879

(22) PCT Filed: May 30, 2001
(86) PCT No.: PCT/GB01/02396

(30) Foreign Application Priority Data
May 30, 2000 (GB) 0013089.8
May 30, 2000 (GB) 0013090.6

(51) Int. Cl. A61K 48/00; A61K 39/395; C12N 15/85

(52) U.S. Cl. 435/455; 514/44; 424/146.1

(57) ABSTRACT

Transfection of confluent cells or other slowly dividing or non-dividing cells that are in contact with each other with a nucleic acid using a non-viral receptor targeted vector may be improved by the concurrent use of an agent that disrupts cell-cell junctions, especially EGTA. The vector is especially an integrin-targeting transfection vector complex comprising (i) a nucleic acid, especially a nucleic acid encoding a sequence of interest, (ii) an integrin-binding component, especially an integrin-targeting peptide, (iii) a polycationic nucleic acid-binding component, especially an oligolysine, and (iv) a lipid component, especially, DOPE, DOTMA, DOSPA or combinations thereof. Various applications of the improved method of transfection are described.

The present invention relates to an improved method of transfecting cells.

The term “transfection” is used herein to denote the introduction of a nucleic acid into a cell. The nucleic acid may be of any origin, and the recipient cell may be prokaryotic or eukaryotic.

Gene therapy and gene vaccination are techniques that offer interesting possibilities for the treatment and/or prophylaxis of a variety of conditions, as does anti-sense therapy. Such techniques require the introduction of a DNA of interest into target cells. The ability to transfer sufficient DNA to specific target cells remains one of the main limitations to the development of gene therapy, anti-sense therapy and gene vaccination. Both viral and non-viral DNA delivery systems have been proposed. In some cases RNA is used instead of DNA.

Receptor-mediated gene delivery is a non-viral method of gene transfer that exploits the physiological cellular process, receptor-mediated endocytosis to internalise DNA. Examples include vectors targeted against insulin receptors, see for example, Rosenkranz et al Experimental Cell Research 199, 323-329 (1992), asialoglycoprotein receptors, see for example, Wu & Wu, Journal of Biological Chemistry 262, 4429-4432 (1987), Chowdhury et al Journal of Biological Chemistry 268, 11265-11271 (1993), and transferrin receptors, see for example, Ciriel et al, Proc. Natl. Acad. Sci. USA 88, 8850-8854 (1991). Further examples of vectors include monoclonal antibodies targeting receptors on neuroblastoma cells (Yano et al, 2000), folate conjugated to liposomes (Reddy & Low 2000, Reddy et al. 1999), galiactose for targeting liver cells (Han et al. 1999 Bettinger et al. 1999) and asialogylcoprotein, also for liver cells (Wu et al. 1991).

Receptor-mediated non-viral vectors have several advantages over viral vectors. In particular, they lack pathogenicity; they allow targeted gene delivery to specific cell types and they are not restricted in the size of nucleic acid molecules that can be packaged. Gene expression is achieved only if the nucleic acid component of the complex is released intact from the endosome to the cytoplasm and then crosses the nuclear membrane to access the nuclear transcription machinery. However, transfection efficiency is generally poor relative to viral vectors owing to endosomal degradation of the nucleic acid component, failure of the nucleic acid to enter the nucleus and the exclusion of aggregates larger than is about 150 nm from clathrin coated vesicles.

Integrins are a super-family of heterodimeric membrane proteins consisting of several different α and β subunits.

They are important for attachment of cells to the extracellular matrix, cell-cell interactions and signal transduction. Integrin-mediated cell entry is exploited for cell attachment and entry by a number of intracellular pathogens including Typanosoma cruzi (Fernandez et al., 1993), adenovirus (Wickham et al., 1993), echovirus (Bergelson et al., 1992) and foot-and-mouth disease virus (Logan et al., 1993) as well as the enteropathogen Y. pseudo-tuberculosis (Isberg, 1991). Egg-sperm fusion is also integrin mediated. Intensive study of the invasin-integrin mediated internalisation process of Yersinia pseudotuberculosis demonstrated that, for efficient cell entry, integrin-binding ligands should have a high binding affinity and a non-polar distribution (Isberg, 1991). Integrin-mediated internalisation proceeds by a phagocytic-like process allowing the internalisation of bacterial cells one to two micrometers in diameter (Isberg, 1991). Targeting of non-viral vectors to integrins, therefore, has the potential to transfect cells in a process that mimics infection of cells by pathogens and avoids the size limitation imposed by clathrin-coated vesicles in receptor-mediated endocytosis.

A further advantage of integrin-mediated vectors is that a large number of peptide ligands for integrin receptors have been described, including sequences derived from natural protein ligands [Verfaille, 1994; Wang, 1995; Staatz, 1991; Pierschbacher, 1984; Massia, 1992, Clements et al. 1994 & Lu et al, 1993] or selected from phage display libraries (Koivunen et al. 1995; 1993; 1994; O'Neil et al. 1992; Healy et al 1995; Pasqualani et al. 1995).

The conserved amino acid sequence arginine-glycine-aspartic acid (RGD) is an evolutionarily conserved feature of many, but not all, natural integrin-binding ligands such as extracellular matrix proteins and viral capsids. Peptides, particularly those containing cyclic-RGD domains can also bind integrins. Peptides containing cyclic-RGD domains are particularly suitable ligands for vectors since they bind to integrins with higher affinities than linear peptides (Koivunen et al. 1995). Hart et al. have demonstrated previously that multiple copies of a cyclic RGD peptide displayed in the major coat protein subunit of fd filamentous phage particles, approximately 900 nm in length, are internalised efficiently by cells in tissue culture in an integrin-mediated manner (Hart et al., 1994). The phage particles were probably internalised by a phagocytic-like process as their size would exclude them from endocytosed vesicles (Hart et al., 1994). Integrin-targeted receptors are found to have

The cyclic RGD-containing peptide GGCRGDMFGCGG[K]₁₆ [SEQ.ID.NO.: 1] was synthesised with a sixteen-lysine tail for complex formation with plasmid DNA (Hart et al., 1995).

Significant levels of integrin-mediated gene expression were achieved in epithelial cell lines with the vector GGCRGDMFGCG[K]₁₆ [SEQ.ID.NO.: 2] (Hart et al., 1995) and the vectors GGCRGDMFGC[K]₁₆ [SEQ.ID.NO.: 3] (WO96/15811). A similar peptide [K]₁₆GACRGDMFGCA [SEQ.ID.NO.: 4], which has the sixteen-lysine domain at the N-terminus and which is easier to syn-thesise than the prototype peptide (WO96/15811 and Hart et al., 1997) generated better transfection levels. Integrin mediated gene expression was generally achieved at levels of about 1 to 10%. The presence of chloroquine in the transfection medium gave some enhancement of transfection in some but not all cell lines tested.

WO 98/54347 discloses a mixture comprising an integrin-binding component, a polycationic nucleic acid-binding component, and a lipid component, and also discloses a complex comprising

(i) a nucleic acid, especially a nucleic acid encoding a sequence of interest,

(ii) an integrin-binding component,

(iii) a polycationic nucleic acid-binding component, and

(iv) a lipid component.

The complex is a transfection vector. The terms “integrin targeting vector”, “integrin targeting complex” and “integrin targeting vector complex” are used interchangeably hereafter to denote the complex described in WO98/53437 and herein. It was observed, surprisingly, that the inclusion of a lipid component in the oligolysine/peptide/DNA complex disclosed in WO96/15811, providing a complex as described in WO98/53437, increases levels of transfection of DNA from about 1 to 10% to about 50 to almost 100%. Not only did the level of transfection increase dramatically but, contrary to previous experience, the increase was observed in all cell lines tested, including endothelial, epithelial and tumour cell lines.

However, all the cell lines tested were transfected under conventional conditions for transfection, that is to say, the cells were sub-confluent. It has been reported that mitosis is required for efficient transformation using cationic liposomes and also when using peptide-based transformation systems (Wilke, 1996; Tseng 1999; Brunner 2000). Confluent cells and/or cells treated with inhibitors of DNA polymerase such as aphidicolin are non-mitotic and transfect with polycationic vectors with low efficiency. The major cell limiting step is considered to be delivery of the transfected nucleic acid into the nucleus. Transportation of the nucleic acid through the cytoplasm to the nucleus is slow and the pores in the nuclear envelope are too small to permit rapid entry of the transfected nucleic acid. It is generally accepted that sub-confluent cultures, in which the cells are dividing, should be used for transfection. We attempted transfection with the transfection complexes of WO98/54347 but using cell line cultures that were confluent i.e. effectively non-mitotic. We found that, even using a transfection complex that had proved previously to be highly efficient for sub-confluent cultures, transfection efficiency did indeed drop dramatically, for example, from about 40 to 50% for sub-confluent cultures to about 5% to 8% for confluent cultures.

EGTA (ethylene glycol-bis(β-aminoethyl ether)-N,N,N′,N′-tetraacetic acid) a calcium chalator (Schmid RW & Reilly CN, Analytical Chemistry 29, 264, 1957) has been described previously as enhancing the gene transfer efficiency of retroviral vectors to airway epithelial cells in vitro (Wang, 1998) and of retroviral and adenoviral vectors in vivo to rabbit tracheal epithelium (Wang, 2000). The proposed mechanism is different for the two viruses, but the role proposed for EGTA is the same: the exposure of viral receptors on the basolateral membranes of the epithelial cells.

However, the use of the calcium-cheating agent EGTA for enhancing transfection using vectors that involve polycation/DNA complexes is contraindicated because it has been shown that calcium ions are important for efficient transformation by polycation/DNA complexes (Haberland, 1999).

The present invention is based on the surprising observation that the use of EGTA with a transfection vector comprising DNA, an integrin-binding component, a polycationic nucleic acid-binding component, and a lipid component, enhances transfection efficiency of confluent cells, which are slowly dividing or non-dividing i.e. substantially non-mitotic cells, in vitro and in vivo. We found that transfection efficiency of airway epithelial cells in vitro using two different reporter genes was increased about four-fold, and in vivo transfection of mouse lungs was also increased about four-fold.

The observation is particularly surprising in view of the fact that calcium ions have previously been shown to be important for transfection using polycationic vectors and, furthermore, the fact that the cells were not mitotic. The use of the calcium-chelating agent EGTA would have been predicted, on the basis of the observations of Haberland, to reduce transfection efficiency, not to increase it. Furthermore, and independently, the problem of access of the introduced nucleic acid to the nucleus still remains. As explained above, the major limiting step in transfection of non-mitotic cells is considered to be delivery of the transfected nucleic acid into the nucleus. Transportation of the nucleic acid through the cytoplasm to the nucleus is slow and the pores in the nuclear envelope are too small to permit rapid entry of the transfected nucleic acid. It should be noted that Wang (1998) used keratinocyte growth factor (KGF) to stimulate proliferation of differentiated epithelia treated with EGTA. No such agent was used in our investigations.

The present invention provides a method of transfecting cells, especially confluent cells or other slowly dividing cells or non-dividing cells that are in contact with each other, that is to say, cells that are substantially non-mitotic, with a nucleic acid, which method comprises treating the cells with a receptor targeted vector comprising the nucleic acid and with an agent that disrupts cell-cell junctions.

The confluent cells or other slowly dividing cells or non-dividing cells that are in contact with each other may be in the form of a culture, for example, a confluent culture in vitro. Methods for producing such cultures are well known, and any cells that can be grown to confluence in vitro may be transfected according to the present invention.

Alternatively, confluent cells or other slowly dividing cells or non-dividing cells that are in contact with each other may be transfected in vivo. A particular example of a tissue that comprises confluent, differentiated and hence substantially non-dividing cells and that has proved recalcitrant to transfection is the airway epithelium, a target for gene therapy for cystic fibrosis and asthma. Other epithelial and endothelial tissues are also particularly suitable targets for transfection according to the present invention.

The cell-cell junctions that are to be disrupted in the method of the present invention are junctions between adjacent cells. The exact types of junction may vary from tissue to tissue to cell type to cell type, and the junctions include gap junctions, tight junctions and adherence junctions. The tight junction, found in epithelia, is also known as zonula occuldens. Tight junctions, located at the apico-lateral border of the epithelial cells, produce the major permeability barrier regulating diffusion of solutes across the epithelium. Tight junctions are calcium-dependent and are formed by a transmembrane protein, occludin, see Anderson (1995) and Mitric (1998). Adherence junctions, located just below the tight junctions, are formed by E-cadherin, a calcium-dependent adhesion molecule essential for the maintenance of cell-cell adhesion, see Takeichi (1990), Takeichi (1991).The cell-cell junctions to be disrupted according to the present invention are not limited to junctions in any specific tissue or between any particular types of cells. Examples of junctions that may be disrupted include those found in certain types of tissues in vivo, for example, epithelial and endothelial tissues.

Agents that are capable of disrupting cell-cell junctions, for example, gap junctions and tight junctions, are known, for example, calcium-chelating agents and calcium-binding agents, for example, EDTA (ethylenediaminetetra-actetic acid) or, especially, EGTA. EGTA is generally preferred as it is more specific for calcium than is EDTA. It is also better tolerated by cells, both in vitro and in vivo.

A further approach is the use of antibodies to substances involved in cell-cell adhesion, for example, at gap junctions, tight junctions, or adherence junctions, for example, cadherins. Antibodies, especially monoclonal antibodies, to such substances, for example, anti-cadherins, may be used as an agent capable of disrupting cell-cell junctions.

The cells are treated with the vector of choice in the usual manner for transfection using that vector. The agent that disrupts cell-cell junctions may be used before the cells are treated with the vector but it is preferably to treat the cells with the agent and the vector at the same time or substantially the same time.

The agent that disrupts cell-cell junctions is used in amount effective to disrupt the junctions. In the case of EGTA for use in vitro, the concentration of EGTA is about 1 mM or less, for example, from about 0.5 mM to 1 mM. Higher concentrations may be used, but care should be taken with regard to toxicity. A concentration of about 1 mM is generally preferred for use in vitro. For use in vivo the concentration of EGTA may be about 25 mM to 200 mM, for example, 100 mM. Concentrations greater than 200 mM may be used, but again care must be taken with regard to toxicity at high concentrations, for example, 400 mM may be lethal is Concentrations of about 100 mM are generally preferred.

The cells may be transfected in vitro or in vivo. Transfection in vitro is particularly useful for transfecting non-dividing cells with genes or anti-sense DNA of interest. Such cells may be harvested for use, for example, for administration to a patient or for use for protein production. Alternatively, such cells may be used in their confluent state, for example, in situ, as disease models for drug testing. It is considered that confluent cells, which are generally non-dividing and may be differentiated, are often better models of tissues that are sub-confluent cells. Cells may be grown to confluence in microwells, or using more sophisticated systems, for example, at an air-liquid interface. Various systems for growing confluent cells for use as models for drug testing are being developed. The ability to transfect cells in such systems is a great advantage.

Confluent cells and other slowly dividing or non-dividing cells that are in contact with each other, including differentiated cells, may be transfected in vivo. The present invention provides such a method of transfection and also provides the use of an agent that disrupts cell-cell junctions and a receptor targeted vector in the manufacture of a medicament for the transfection of cells, especially confluent, or other slowly dividing or non-dividing cells that are in contact with each other, for example, substantially non-mitotic cells.

Such cells include, for example, endothelial or epithelial cells, for example, cells of the any part of the airway epithelium, including bronchial and lung epithelium, and the corneal endothelium. The airway epithelium is an important target for gene therapy for cystic fibrosis and asthma.

The vector that may be used in the present invention is in particular any non-viral vector that is targeted to a cell-surface receptor. As mentioned above, many such vectors have been proposed, including vectors targeted to insulin, asialoglycoprotein and transferrin receptors, see above. Further examples of vectors include monoclonal antibodies targeting receptors on neuroblastoma cells (Yano et al, 2000), folate conjugated to liposomes (Reddy & Low 2000, Reddy et al. 1999), galactose for targeting liver cells (Han et al. 1999 Bettinger et al. 1999) and asialogylcoprotein, also for liver cells (Wu et al. 1991). It may be advantageous to use a vector that comprises a polycationic component.

Vectors targeted against integrin receptors have particular advantages, especially the vectors described in WO96/15811 and, in particular, the transfection vector complexes described in WO98/54347 and herein.

Whatever vector is used, the nucleic acid may be obtained from natural sources, or may be produced recombinantly or by chemical synthesis. It may be modified, for example, to comprise a molecule having a specific function, for example, a nuclear targeting molecule. The nucleic acid may be DNA or RNA. DNA may be single stranded or double stranded. The nucleic acid may be suitable for use in gene therapy, in gene vaccination or in an anti-sense therapy. The nucleic acid may be or may relate to a gene that is the target for particular gene therapy that is to say, a gene for newer treatment by gene therapy is desired, fore example, a gene having a mutation, or another defect, or a gene that is deficient, or a gene that is deficient, or a gene that is absent or is present in insufficient amounts or that is present in excess, any of which effects causes a disease or disorder. The nucleic acid may be a molecule that can function as a gene vaccine or as an anti-sense therapeutic agent. The nucleic acid may be or correspond to a complete coding sequence or may be part of a coding sequence or may be a control or regulatory element or may be or correspond to a genetic sequence comprising all or some elements selected from the coding sequence of a gene and the upstream and downstream non translated sequences, including control and regulatory elements.

Alternatively, the nucleic acid may encode a protein that is commercially useful, for example industrially or scientifically useful, for example an enzyme; pharmaceutically useful, for example, a protein that can be used therapeutically or prophylactically as a medicament or vaccine; or diagnostically useful, for example, an antigen for use in an ELISA. Host cells capable of producing commercially useful proteins are sometimes called “cell factories”.

Appropriate transcriptional and translational control elements are generally provided when the nucleic acid is a protein coding sequence. For gene therapy, the nucleic acid component is generally presented in the form of a nucleic acid insert in a plasmid or vector. In some cases, however, it is not necessary to incorporate the nucleic acid component in a vector in order to achieve expression. For example, gene vaccination and anti-sense therapy can be achieved using a naked nucleic acid.

The nucleic acid is generally DNA but RNA may be used in some cases, for example, in cancer vaccination. The nucleic acid component may be referred to below as the plasmid component or component “D”.

The receptor targeted vector is especially an integrin targeted complex as described in WO98/54347, that is to say, a complex comprising

(i) a nucleic acid, especially a nucleic acid encoding a sequence of interest,

(ii) an integrin-binding component,

(iii) a polycationic nucleic acid-binding component, and

(iv) a lipid component.

According to WO98/54347, the integrin-binding component is any component that is capable of binding specifically to integrins found on the surface of cells. The integrin-binding component may be a naturally occurring integrin-binding ligand, for example, an extracellular matrix protein, a viral capsid protein, the bacterial protein invasin, a snake venom disintegrin protein, or an integrin-binding fragment of any such protein. Such integrin-binding proteins and fragments thereof may be obtained from natural sources or by recombinant techniques, but they are difficult to synthesise and purify in large amounts, they require conjugation directly to DNA or RNA or to polycationic elements for DNA or RNA binding, and are immunogenic in vivo.

It is preferable to use integrin-binding peptides, in particular because of their ease of synthesis, purification and storage, their potential for chemical modification, and their potentially low immunogenicity in vivo. Examples of integrin-binding peptides are given in Verfaille, 1994 #635; Wang, 1995 #645; Staatz, 1991 #539; Pierschbacher, 1984 #314; Massia, 1992 #86, Clements et al. 1994 & Lu et al, 1993; and in Koivunen et al. 1995; 1993; 1994; O'Neil et al. 1992; Healy et al 1995; and Pasqualani et al. 1995.

As indicated above, peptides containing the conserved amino acid sequence arginine-glycine-aspartic acid (RGD) bind with high affinity to integrins. Accordingly, peptides comprising the RGD sequence are particularly useful. The affinity between integrin and peptide ligands is influenced by the amino acid sequence flanking the RGD domain. In peptides having a cyclic region encompassing all or part of the region comprising the RGD sequence, the conformational freedom of the RGD sequence is restricted. Such peptides generally have a higher affinity for integrin receptors than do their linear counterparts. Such cyclic peptides are particularly preferred. Cyclic peptides may be formed by the provision of two cysteine residues flanking the RGD sequence in the peptide, thus enabling the formation of a disulphide bond. A cysteine residue may be separated from the RGD sequence by one or more residues, for example, up to six residues, or may be immediately adjacent to the RGD sequence, although preferably both cysteines are not immediately adjacent to the ends of the RGD sequence. Two further cysteine residues may be present, enabling formation of two disulphide bonds.

An example of an amino acid sequence that will permit cyclisation by disulphide bond formation is CRGDMFGC [SEQ.ID.NO.: 5]. A peptide that consists of or comprises the sequence CRGDMFGC may advantageously be used as an integrin-binding peptide according to the present invention. Examples of peptides that comprises the sequence CRGDMFGC and that are effective integrin-binding ligands are the peptides GGCRGDMFGC [SEQ.ID.NO.: 6], GGCRGDMFGCG [SEQ.ID.NO.: 7], GGCRGDMFGCA [SEQ.ID.NO.: 8] and GACRGDMFGCA [SEQ.ID.NO.: 9].

The peptide GACDCRGDCFCA [SEQ.ID.NO.: 10] has the potential to form two disulphide bonds for stabilising the RGD loop. That peptide and others having the potential to form two RGD-stabilising disulphide bonds by the presence of two or more cysteine residues, may be particularly useful as integrin-binding ligands according to the present invention.

However, not all integrin-binding peptides contain the conserved RGD sequence. For example, the peptides GACRRETAWACA [SEQ.ID.NO.: 11] and GACRRETAWACG [SEQ.ID.NO.: 12] are integrin-specific peptides. Other peptides comprising the sequence CRRETAWAC [SEQ.ID.NO.: 13] may be used, as may other non-RGD peptides, particularly those that have the potential for disulphide bond formation by the provision of two or more cysteine residues.

Peptide sequences may be designed on the basis of known ligands, for example, on the basis of integrin-binding domains of naturally-occurring integrin-binding ligands, or on the basis of known peptides that bind to integrins.

As stated above integrins are a family of heterodimeric proteins found on the surface of cells. They consist of several different α and β subunits. Some integrins are found on may types of cells, others are more specific, for example, α5 and αv integrins are widespread and are found on a diverse range of cells. Integrin-binding ligands can vary in their affinity for different integrins. For example, GACRGDMFGCA [SEQ.ID.NO.: 9] (peptide 1) has affinity for α5 and αv integrins but is non-specific (O'Neil et al. 1992, Hart et al. 1997). GACDCRGDCFCA [SEQ.ID.NO.: 10] (peptide 5) has high affinity for integrin αv but is not αv-specific (Koivunen et al. 1995; Hart et al. 1997). GACRRETAWACG [SEQ.ID.NO.: 11] (peptide 6) however, which does not contain the conserved RGD region, is α5β1-specific (Koivunen et al. 1995). Various integrin-binding peptides and their integrin specificity are set out in the Table below:

TABLE

| Peptide | | |
|---|---|---|
| number and | | |
| integrin | | |
| specificity | Sequence | SEQ.ID.NO. |
| Peptide 1 | GACRGDMFGCA | SEQ.ID.NO.:9 |
| (αv,α5β1) | | |
| Peptide 2 | GACRGDMFGCGG | SEQ.ID.NO.:15 |
| (αv,α5β1) | | |
| Peptide 5 | GACDCRGDCFCA | SEQ.ID.NO.:10 |
| (αv) | | |
| Peptide 6 | GACRRETAWACG | SEQ.ID.NO.:12 |
| (α5β1) | | |
| Peptide 7 | GAGPEILDVPST | SEQ.ID.NO.:16 |
| (α4β1) | | |
| Peptide 8 | GACQIDSPCA | SEQ.ID.NO.:17 |
| (α4β1) | | |
| Peptide 9 | GACRRETAWACGKGACRRETAWACG | SEQ.ID.NO.:18 |
| (α5β1) | | |

In the various peptides described above by sequence, the inital two residues “GG” or “GC”, where present, are spacers. A preferred embodiment of the present invention is based on the observation that a modified spacer has improved transfection efficiency.

Accordingly, in the method of the present invention the receptor-targeted vector is preferably a complex that comprises

(i) a nucleic acid, especially a nucleic acid encoding a sequence of interest,

(ii) an integrin-binding component,

(iii) a plycationic nucleic acid-binding component, and

(iv) a lipid component,in which complex the integrin binding component comprises an integrin-binding element and a spacer element, the spacer element being longer and/or more hydrophobic than the dipeptide spacers GG (glycine-glycine) and GA (glycine-alanine).

Such a preferred spacer element is generally a peptide, that is to say, it comprises amino acid residues. The amino acids may be naturally occurring or non-naturally occurring. They may have L- or D-configuration.

A preferred spacer element may be longer than a dipeptide. It may, for example, comprise three or more amino acids, for example, four or more, for example, five or more, for example, up to ten amino acids or more. The amino acids may be the same or different, but the use of multiple lysine residues should be avoided in the preferred spacer as oligolysine sequences are the preferred polycationic nucleic acid-binding component of a complex of the present invention.

The preferred spacer may be more hydrophobic than the dipeptides GG and GA. For example, amino acids that are more hydrophobic than glycine and alanine may be used. Examples of hydrophobic amino acids are well known and include E-amino hexanoic acid. A preferred spacer may be either longer or more hydrophobic than the dipeptides GG and GA, or it may be both longer and more hydrophobic.

An example of the latter type of spacer is XSXGA [SEQ.ID.NO. : 14] wherein S=serine, G=glycine, A=alanine and X=ε-amino hexanoic acid. This spacer is higly hydrophobic.

A spacer, whether GA or GG, or a preferred spacer as described above, is generally at the N-terminus of the integrin-binding peptide.

It will be appreciated that in many cases the “integrin binding peptides” described above comprise both an integrin-binding peptide element and a spacer dipeptide GG or GC, see for example, the peptides of GGCRGDMFGC SEQ.ID.NO: 6, which comprises the integrin-binding element CRGDMFGC [SEQ.ID.NO.: 5]and the dipeptide spacer GG. The preferred spacer element of the present invention takes the place of a GG or GA spacer in peptides described above.

For example, integrin binding peptides include the following: CRGDMFGC [SEQ.ID.NO.: 27]; CRGDMFGCG [SEQ.ID.NO.: 5]; CRGDMFGCA [SEQ.ID.NO.: 28]; CDCRGDCFCA [SEQ.ID.NO.: 29]; CRRETAWACA [SEQ.ID.NO.: 30]; CRRETAWAC [SEQ.ID.NO.: 13]; CRRETTAWAC [SEQ.ID.NO.: 31]; CRRETAWACG [SEQ.ID.NO.: 32]; CRGDMFGCGG [SEQ.ID.NO.: 33]; GPEILDVPST [SEQ.ID.NO.: 34]; CQIDSPCA [SEQ.ID.NO.: 35]; CRRETAWACGKGACRRETAWACG [SEQ.ID.NO.: 36]. Further suitable peptides are described, for example, in WO95/14714.

A preferred spacer may be linked to any of the above peptides, preferably at the or terminus thereof. The terms “integrin-binding component” and “integrin-binding peptide” and any other such term as used herein includes all integrin binding components described herein, including those with spacer elements GG and GA and those with preferred spacer elements.

The polycationic nucleic acid-binding component is any polycation that is capable of binding to DNA or RNA is retained. For example, from 4 to 100 cationic monomers may be present, for example, from 10 to 20, especially about 16. An oligolysine is particularly preferred, for example, having from 3 to 100 lysine residues, for example, from 10 to 20, for example, from 13 to 19, for example, from 14 to 18, for example, from 15 to 17 residues, especially 16, 17 or 18 residues especially [K]₁₆, “K” denoting lysine.

The polycationic DNA or RNA-binding component may advantageously be linked or otherwise attached to the integrin-binding component. A combined integrin-binding component/polycationic DNA or RNA-binding component may be referred to below as component “I”. For example, a polycationic DNA or RNA-binding component may be chemically bonded to an integrin-binding component, for example, by a peptide bond in the case of an oligolysine. The polycationic component may be linked at any position of the integrin-binding component. Preferred combinations of integrin-binding component and polycationic DNA or RNA-binding component are an oligolysine, especially [K]₁₆, linked via a peptide bond to a peptide, for example, a peptide as described above.

A combined integrin-binding component/polycationic DNA or RNA-binding component is a component having an integrin-binding moiety that is the integrin-binding component as defined and described herein linked or otherwise attached to a polycationic DNA or RNA binding moiety that is a polycationic DNA or RNA component as defined and described herein.

It is disclosed in WO98/54347 that the use of a lipid greatly enhances transfection for all peptides and all cell types tested, unlike other enhancement techniques that have been tried, for example, chloroquine, which enhance transfection to a small extent in some but not all cell types tested.

The lipid component may be or may form a cationic liposome. The lipid component may be or may comprise one or more lipids selected from cationic-lipids and lipids having membrane destabilising or fusogenic properties, especially a combination of a cationic lipid and a lipid that has membrane destabilising properties.

A preferred lipid component (“L”) is or comprises the neutral lipid dioleyl phosphatidylethanolamine, referred to herein as “DOPE”. DOPE has membrane destabilising properties sometimes referred to as “fusogenic” properties (Farhood et al. 1995).

Other lipids, for example, neutral lipids, having membrane destabilising properties, especially membrane destabilising properties like those of DOPE may be used instead of or as well as DOPE.

Other phospholipids having at least one long chain alkyl group, for example, di(long alkyl chain)phospholipids may be used. The phospholipid may comprise a phosphatidyl group, for example, a phosphatidylalkanolamine group, for example, a phosphatidyl-ethanolamine group.

A further preferred lipid component is or comprises the cationic lipid N-[1-(2,3-dioleyloxy)propyl]-N,N,N-trimethyl-ammonium chloride, referred to herein as “DOTMA”. DOTMA has cationic properties. Other cationic lipids may be used in addition to or as an alternative to DOTMA, in particular cationic lipids having similar properties to those of DOTMA. Such lipids are, for example, quaternary ammonium salts substituted by three short chain alkyl groups, and one long chain alkyl group. The short chain alkyl groups may be the same or different, and may be selected from methyl and ethyl groups. At least one and up to three of the short chain alkyl group may be a methyl group. The long alkyl chain group may have a straight or branched chain, for example, a di(long chain alkyl)alkyl group.

Another preferred lipid component is or comprises the lipid 2,3-dioleyloxy-N-[2-(spermidinecarboxamido)ethyl]-N,N-dimethyl-1-propanaminiumtrifluoridoacetate, referred to herein as “DOSPA”. Analogous lipids may be used in addition to or as an alternative to DOSPA, in particular lipids having similar properties to those of DOSPA. Such lipids have, for example, different short chain alkyl groups from those in DOSPA.

A preferred lipid component comprises DOPE and one or more other lipid components, for example, as described above. Especially preferred is a lipid component that comprises a mixture of DOPE and DOTMA. Such mixtures form cationic liposomes. An equimolar mixture of DOPE and DOTMA is found to be particularly effective. Such a mixture is known generically as “lipofectin” and is available commercially under the name “Lipofectin”. The term “lipofectin” is used herein generically to denote an equimolar mixture of DOPE and DOTMA. Other mixtures of lipids that are cationic liposomes having similar properties to lipofectin may be used. Lipofectin is particularly useful as it is effective in all cell types tested.

A further preferred lipid component comprises a mixture of DOPE and DOSPA. Such mixtures also form cationic liposomes. A mixture of DOPE and DOSPA in a ratio by weight 3:1 DOSPA:DOPE is particularly effective. Such a mixture, in membrane filtered water, is available commercially under the name “Lipofectamine”. Mixtures comprising DOPE, DOTMA and DOSPA may be used, for example, mixtures of lipofectin and lipofectamine.

Other cationic lipids are available commercially, for example, DOTAP (Boehringer-Mannheim) and lipids in the Tfx range (Promega). DOTAP is N-[1-(2,3-diolyloxy)propyl]-N,N,N-trimethylammonium methylsulphate. The Tfx reagents are mixtures of a synthetic cationic lipid [N,N,N′,N′-tetramethyl-N,N′-bis(2-hydroxyethyl)-2,3-di(oleoyloxy)-1,4-butanediammonium iodide and DOPE. All the reagents contain the same amount of the cationic lipid component but contain different molar amounts of the fusogneic lipid, DOPE.

However, lipofectin and lipofectamine appear to be markedly more effective as the lipid component in LID complexes of the present invention than are DOTPA and Tfx agents.

The effectiveness of a putative integrin-binding component, polycationic DNA or RNA-binding component, or of lipid component may be determined readily using the methods described herein.

The efficiency of transfection using an integrin-targeted transfection complex as described above as transfection vecotr is influenced by the ratio lipid component:integrin-binding component:DNA or RNA. For any chosen combination of components for any particular type of cell to be transfected, the optimal ratios can be determined simply by admixing the components in different ratios and measuring the transfection rate for that cell type, for example, as described herein.

For example, a combination consisting of a pGL2 plasmid, which is a plasmid encoding luciferase (a reporter gene) under an SV40 promoter as DNA component (D), [K]₁₆GACRGDMFGCA [SEQ.ID.NO.: 20] ([K]₆-peptide 1) as a combined integrin-binding component/polycationic DNA binding component (I), and lipofectin (DOPE:DOTMA 1:1 molar ratio) as the lipid component (L) was tested to find the optimal ratio of components. Complexes formed with 1 μg of lipofectin (L) and 4 μg of [K)₁₆-peptide (I) per 1 μg of plasmid (D) were 100-fold more active than complexes lacking lipofectin. Addition of larger amounts of lipofectin reduced transfection activity in a lipofectin dosedependent manner.

An optimal transfection ratio of 0.75 μg of lipofectin (L) per 4 μg of the [K]₃₆-peptide integrin-binding component/-polycationic DNA or RNA-binding component (I) per 1 μg plasmid DNA or RNA (nucleic acid component, D) was found for three different cell lines namely melanoma cell, endothelial cells and epithelial cells. That ratio was subsequently found to be effective for other different cell lines and for other oligolysine-peptides. A ratio L:I:D of 0.75:4:1 by weight corresponds to a molar ratio of 0.5 nmol lipofectin: 1.25 nmol [K]₁₆-peptide 6: 0.25 pmol plasmid pGL2-control. A ratio L:I:D of 0.75:4:1 by weight, or the corresponding molar ratio are preferred when lipofectin is used as the lipid component.

For a combination of components in which lipofectin is replaced by lipofectamine (DOPE/DOSPA), the optimal ratio was found to be 12 μg lipofectamine: 4 μg [K]₁₆-peptide 6: 1 μg plasmid DNA or RNA. A ratio of L:I:D of 12:4:1 by weight, or the corresponding molar ratio, is appropriate for lipofectamine-containing complexes. Optimal ratios for other systems may be determined analogously.

Lipofectin and lipofectamine appear to be particularly effective in enhancing transfection in the system described above. Lipofectin has the advantage that only very small amounts are required. Any side effects that may occur are therefore minimised. As indicated above, the optimal weight ratio of components L:I:D when using lipofectamine is 12:4:1. With lipofectin the optimal ratio is only 0.75:4:1.

A transfection complex as described above may be produced by admixing components (i), (ii), (iii) and (iv).

Although the components may be admixed in any order, it is generally preferable that the lipid component is not added last. In the case where there is a combined integrin-binding component/polycationic DNA or RNA-binding component it is generally preferable to combine the components in the following order: lipid component; combined integrin-binding/polycationic DNA or RNA-binding component; DNA or RNA component, for example, in the order: lipofectin, oligolysine-peptide component, DNA or RNA component.

For use with EGTA in vivo, it is preferable to mix the components as follows: mix the lipid component with the integrin binding/polycationic DNA or RNA binding component; mix the DNA or RNA with the EGTA; mix the two mixtures.

A transfection mixture comprising an integrin-binding component, a polycationic nucleic acid-binding component, and a lipid component may be used to produce a nucleic acid-containing transfection complex as described above by the incorporation of a nucleic acid with the mixture, for example, by admixture. Alternatively, the transfection mixture may be used for the production of a complex which comprises, instead of the nucleic acid component, any other component that is capable of binding to the polycationic nucleic-acid binding component, for example, a protein.

The individual components of a transfection mixture of the invention are each as described above in relation to the transfection complex. The preferred components, preferred combinations of components, preferred ratios of components and preferred order of mixing, both with regard to the mixture and to the production of a complex, are as described above in relation to the transfection complex.

A transfection mixture preferably comprises an equimolar mixture of DOPE and DOTMA (lipofectin) as the lipid component and an oligolysine-peptide especially a [K]₁₆-peptide as a combined integrin-binding/nucleic acid-binding component. The preferred molar ratio lipofectine:oligolysine-peptide is 0.75:4.

As indicated above, the present invention provides a method of transfecting cells, especially confluent cells or other slowly dividing or non-dividing cells that are in contact with each other, with a nucleic acid, which comprises contacting the cells in vitro or in vivo with a receptor-targeted vector comprising a nucleic acid and with an agent that disrupts cell-cell junctions under conditions suitable for effecting transfection.

The present invention also provides a process for expressing a nucleic acid in host cells, especially confluent cells or other slowly dividing or non-dividing cells that are in contact with each other, which comprises contacting the host cells in vitro or in vivo with a receptor-targeted vector comprising the nucleic acid and with an agent that disrupts cell-cell junctions under conditions suitable for effecting transfection and then culturing the host cells under conditions that enable the cells to express the nucleic acid.

The present invention further provides a process for the production of a protein in host cells, especially confluent cells or other slowly dividing or non-dividing cells that are in contact with each other, which comprises contacting the host cells in vitro or in vivo with a receptor-targeted vector that comprises a nucleic acid that encodes the protein and with an agent that disrupts cell-cell junctions, under conditions suitable for effecting transfection, allowing culturing the host cells under conditions suitable for protein production, the cells to express the protein, and obtaining the protein. The protein may be obtained either from the host cell or from the culture medium.

The present invention further provides confluent cells or other slowly dividing or non-dividing cells that are in contact with each other, transfected with a nucleic acid, and also the progeny of such cells.

The present invention further provides a disease model for use in testing candidate pharmaceutical agent, which comprises confluent cells or other slowly dividing or non-dividing cells that are in contact with each other, transfected with a nucleic acid suitable for creating the disease model.

The present invention also provides a pharmaceutical composition which comprises(i) a receptor-targeted vector comprising a nucleic acid and (ii) an agent that disrupts cell-cell junctions, in admixture or conjunction with a pharmaceutically suitable carrier. The composition may be a vaccine.

The present invention also provides a method for the treatment or prophylaxis of a condition caused in a human or in a non-human animal by a defect and/or a deficiency in a gene, which comprises administering to the human or to the non-human animal (i) a receptor-targeted vector comprising a nucleic acid suitable for correcting the defect or deficiency and (ii) an agent that disrupts cell-cell junctions in an amount effective for said treatment or prophylaxis.

The present invention also provides a method for therapeutic or prophylactic immunisation of a human or of a non-human animal, which comprises administering to the human or to the non-human animal (i) a receptor-targeted vector comprising an appropriate nucleic acid and (ii) an agent that disrupts cell-cell junctions in an amount effective for said therapeutic or prophylactic immunisation.

The present invention also provides a method of anti-sense therapy of a human or of a non-human animal, comprising anti-sense DNA administering to the human or to the non-human animal (i) a receptor-targeted vector comprising the anti-sense nucleic acid and (ii) an agent that disrupts cell-cell junctions in an amount effective for said anti-sense therapy.

The present invention also provides the use of (i) a receptor-targeted vector comprising a nucleic acid and (ii) an agent that disrupts cell-cell junctions for the manufacture of a medicament for the prophylaxis of a condition caused in a human or in a non-human animal by a defect and/or a deficiency in a gene, for therapeutic or prophylactic immunisation of a human or of a non-human animal, or for anti-sense therapy of a human or of a non-human animal.

A non-human animal is, for example, a mammal, bird or fish, and is particularly a commercially reared animal.

The nucleic acid, either DNA or RNA, in the vector is appropriate for the intended use, for example, for gene therapy, gene vaccination, or anti-sense therapy. The DNA or RNA and hence the vector is administered in an amount effective for the intended purpose. Conditions suitable for effecting transfection and for allowing cells to express protein are well known. Suitable conditions are described, for example, in the following Examples.

The treatments and uses described above may be carried out by administering the respective vector, agent or medicament in an appropriate manner, for example, administration may be topical, for example, in the case of airway epithelia.

In a further embodiment, the present invention provides a kit comprising (i) a receptor-targeted vector comprising a nucleic acid and (ii) an agent that disrupts cell-cell junctions.

The present invention also provides a kit that comprises an agent that disrupts cell-cell junctions and the following items: (a) an integrin-binding component; (b) a polycationic nucleic acid-binding component, and (c) a lipid component. Such a kit may further comprise (d) either a nucleic acid or a plasmid or vector suitable for the expression of a nucleic acid, the plasmid or vector being either empty or comprising the nucleic acid.

The components (a) to (d) kit are, for example, as described above in relation to a integrin-targeted transfection complex or a mixture as described above.

A kit generally comprises instructions, which preferably indicate the preferred ratios of the components and the preferred order of use or admixing of the components, for example, as described above. A kit may be used for gene therapy, gene vaccination or anti-sense therapy. Alternatively, it may be used for transfecting a host cell with a nucleic acid encoding a commercially useful protein i.e. to produce a so-called “cell factory”.

In a kit of the invention the components including the preferred components are, for example, as described above in relation to a complex of the present invention.

The present invention also provides a method for increasing the efficiency of transfecting confluent cells or other slowly dividing or non-dividing cells that are in contact with each other with a receptor targeted vector, which comprises treating the cells with an agent that disrupts cell-cell junctions.

In each case, the vector and the agent that disrupts cell-cell junctions are each as described above. The vectors are non-viral vectors. The confluent cells or other slowly dividing or non-dividing cells that are in contact with each other are cells that are substantially non-mitotic.

The vector is especially an integrin targeted transfection vector complex, as described above and in WO98/53437, in particular one of the preferred complexes. The integrin-binding component is preferably targeted at an integrin that is expressed abundantly in the tissue of interest. For example, α5β1 integrins are expressed abundantly on bronchial epithelial cells, so an integrin-binding component directed at α5β1 integrins is preferably used for transfection of such airway cells. The polycationic nucleic acid binding component is preferably an oligolysine, as described above. The lipid component is preferably capable of forming a cationic liposome, and preferably is or comprises DOPE and/or DOTMA, for example, an equimolar mixture thereof, or is or comprises DOSPA, for example, a mixture of DOPE and DOSPA, for example in the weight ratio DOPE:DOSPA of 1:3. The rations between the components are preferably as described above, as is the order of mixing of the components.

The agent that disrupts cell-cell junctions is especially a calcium-binding agent, especially EGTA or maybe an antibody to a substance involved in cell-cell adhesion, for example, an anti-cadherin.

Targets for gene therapy are well known and include monogenic disorders, for example, cystic fibrosis, various cancers, and infections, for example, viral infections, for example, with HIV. For example, transfection with the p53 gene offers great potential for cancer treatment. Targets for gene vaccination are also well known, and include vaccination against pathogens for which vaccines derived from natural sources are too dangerous for human use and recombinant vaccines are not always effective, for example, hepatitis B virus, HIV, HCV and herpes simplex virus. Targets for anti-sense therapy are also known. Further targets for gene therapy and anti-sense therapy are being proposed as knowledge of the genetic basis of disease increases, as are further targets for gene vaccination.

Many of those targets are tissues comprising confluent cells or other slowly dividing or non-dividing cells that are in contact with each other i.e. cells that are substantially non-mitotic. The present invention enhances the transfection efficiency and hence the effectiveness of the treatment.

Transfection vector complexes described in WO98/53437 and above have been demonstrated to transfect various different cell types, including endothelial and epithelial cells, and tumour cells. Transfection of all cell types tested including cell types that are particularly resistant to transfection with most plasmid transfection vectors, for example, neuroblastoma cells, primary smooth muscle cells and cardiac myocytes, and haematopoieic cells has been achieved with high efficiency using transfection complexes of the present invention. This enables effective gene therapy, gene vaccination and anti-sense therapy without the previous restrictions as to cell type. For example, transfection with the p53 gene for cancer therapy has great potential but is currently limited by the range of cell types in which effective transfection can be achieved. The present invention enhances the transfection efficiency of such vectors when used to transfect confluent cells or other slowly dividing or non-dividing cells that are in contact with each other and hence the effectiveness of the treatment.

The effective transfection of neuroblastoma cells demonstrates that the complexes of the invention may be used as vaccines or for therapy of neuroblastoma, an important childhood malignancy. The effective transfection of primary smooth muscle cells and cardiac myocytes, which are particularly resistant to plasmid-mediated transfection, demonstrates that diseases and other pathological conditions affecting muscles and the cardiovascular system can now be treated by gene therapy. One such condition is restenosis. After balloon angioplasty plaques reform in 30-50% of cases. A gene that prevents proliferation of cells in blood vessel walls may be introduced using a complex of the present invention to reduce restenosis.

Haematopoietic cells are another cell type that is particularly resistant to plasmid-mediated transfection. The effectiveness of tranfection using a complex of the present invention, which can exceed 60%, now enables gene therapy, gene vaccination and anti-sense therapy of diseases involving haematopoietic cells, including leukaemia and bone marrow stem cell disorders. For example, transfection of a cytokine gene may be used for adjuvant immunotherapy.

Complexes of the invention have been demonstrated to be effective vectors for intracellular transport and delivery of anti-sense oligonucleotides, which enables antiviral and cancer therapy.

Furthermore, complexes of the invention have been demonstrated to be effective for intracellular transport of very large DNA molecules, for example, DNA larger than 125 kb, which is particularly difficult using conventional vectors. This enables the introduction of artificial chromosomes into cells.

Transfection at high levels has been demonstrated in vivo, confirming the utility of the complexes of the invention for gene therapy, antisense therapy and gene vaccination.

In all cases, the present invention enhances the transfection efficiency and hence the effectiveness of the treatment when confluent cells or other slowly dividing or non-dividing cells that are in contact with each other are treated.

Transfection of the airways, for example, the bronchial epithelium demonstrates utility for gene therapy of, for example, cystic fibrosis and asthma. Transfection of corneal endothelium demonstrates utility for treatment of eye disease affecting the cornea or corneal organ transplants, for example in glaucoma. The present invention is particularly useful for enhancing the transfection efficiency and hence the effectiveness of the treatment of such epithelial and endothelial tissues.

The enhanced levels of transfection make the method of the invention particularly suitable for the production of host cells capable of producing a desired protein, so-called “cell factories”. For long-term production, it is desirable that the introduced nucleic acid is incorporated in the genome of the host cell, or otherwise stably maintained. That can be readily ascertained. As indicated above, the range of proteins produced in this way is large, including enzymes for scientific and industrial use, proteins for use in therapy and prophylaxis, immunogens for use in vaccines and antigens for use in diagnosis.

Confluent cells may be used as tissue models, for example, of epithelial or endothelial tissue, for example, for use in testing of drugs. The ability to achieve effective transfection of such cells according to the present invention using greatly increases the potential uses of confluent cell cultures as disease models. Such transfected cell cultures are part of the present invention, as are their uses in drug testing.

Accordingly, the present invention provides a method of testing drugs in a tissue model for a disease, wherein the tissue model comprises transgenic confluent cells obtained by transfecting confluent cells with a nucleic acid by contacting the cell with a receptor-targeted vector comprising a nucleic acid and with an agent that disrupts cell-cell junctions.

The present invention is especially useful with a receptor targeted vector that is capable of high efficiency targeted vector that is capable of high efficiency transfection. In a preferred embodiment, the vector comprises four modular elements; an oligolysine, especially [K]₁₆, DNA or RNA-binding element; a high affinity integrin-binding peptide, for example, a peptide described herein; a DNA or RNA sequence, optionally in a plasmid, and optionally regulated by a viral promoter and an enhancing element; the cationic liposome DOTMA/DOPE (lipofectin). The combination of oligolysine-peptide/DNA or RNA complex with the cationic liposome formulation DOTMA/DOPE is a potent combination. Alternatively a DOPE/DOSPA formulation may be used instead of or in addition to a DOTMA/DOPE formulation. The optimisation of variables associated with complex formation and the mode of transfection by LID complexes has been demonstrated. In addition, analysis by atomic forces microscopy has been carried out to assess the structure of the complexes. The most important variables in the formation of optimal LID transfection complexes appear to be the ratio of the three components and their order of mixing. The same composition appears to be optimal for all cell lines tested.

The mechanism of action of the LID complex, the reason for the unexpectedly high levels of transfection and the surprisingly wide variety of cells that can be transfected at that high efficiency are not yet understood.

However, the following observations set out in WO98/53437 indicate that the role of the lipid component is to enhance the efficiency of transfection mediated by oligolysine-peptide/DNA or RNA complexes:

The level of transfection with LID (lipofectin/[K]₁₆-peptide/plasmid) complexes is three to six fold higher than that with LKD (lipofectin/(K]₁₆/plasmid) complexes prepared with the same charge ratios, or with LD (lipofectin/plasmid) complexes. This indicates that the integrin-targeting moiety, i.e. the peptide, is a significant factor in the transfection efficiency of those complexes.

Optimised LID transfection complexes contain only one seventh of the amount of lipofectin required for optimal transfection with LD complexes. Transfections with low-ratio LD complexes that contain the same ratio of lipofectin to [K]₁₆-peptide/-plasmid as in optimal LID complexes but no [K]₁₆-peptide, did not transfect cells at all. This suggests that the role of lipofectin in LID complexes is to enhance transfection mediated by the integrin receptor-binding peptide.

Furthermore, we have found that both LID and ID complexes both form spherical particles of similar sizes. Optimal LD complexes, however, formed a tubular network with some tubule-associated particles, which suggests a different type of cellular interaction and transfection mechanism from LID and ID transfections.

It is possible that condensation of plasmid DNA or RNA by the oligolysine element of the integrin-targeting oligolysine-peptides and the cationic charge of the complexes may lead to high levels of expression when associated with lipofectin, and the integrin targeting moiety i.e. the peptide is irrelevant. Transfection experiments with LKD complexes, mixed in the same order and the same charge ratios as the LID complexes, were more efficient than LD or KD complexes. To assess the contribution of the relative importance of the oligolysine element and the integrin-targeting peptide domain of the combined integrin-binding component/polycationic DNA or RNA-binding component I, transfection by LID complexes were prepared containing a range of proportions of [K]₁₆ and [K]₁₆integrin targeting peptide 6, [K]₁₆GACRRETAWACG [SEQ.ID.NO.: 21]. Transfection expression data indicate higher efficiencies with complexes in which increasing amounts of [K]₁₆peptide 6 replace [K]₁₆ and a dose-dependency on the amount of integrin-targeting (ligandbinding) domain i.e. peptide 6.

The ratio of components mixed together to form the optimal transfection complex is also informative as to the possible mechanism of lipofectin mediated enhancement. The DOTMA element of lipofectin is cationic, which may enhance the activity of the complex, while DOPE may have the ability to destabilise the endosomal membrane (Farhood et al., 1995) enhancing endosomal release of plasmid DNA or RNA. The components of the LID complexes are mixed together in constant optimal ratios. It is assumed that the particles formed also contain these elements in the same proportions. Therefore, 3 nmol negative charge from plasmid DNA or RNA are associated with approximately 21 nmol positive charge from the [K]₁₆peptide. Lipofectin, however, provides only a further 0.25 nmol of positive charge. This suggests that, contrary to expectations, the enhancing effect of lipofectin in LID complexes is not charge related but may relate to the membrane destabilising effect of the DOPE component.

While not limited to the following theory of the mechanism of action, the following model of the early stages of the transfection process, which is based on the observations described herein, is proposed to explain the surprising and unexpected high efficiency of transfection by LID complexes, which high efficiency is found in all the cell types investigated.

The complexes are formed electrostatically by random association of lipofectin, oligolysine-peptide and plasmid DNA or RNA. The relative high proportion of oligolysine-peptide ensures a high proportion of integrin-targeting ligands per plasmid molecule. Particles are formed that contain one or more plasmids, associated with thousands of oligolysine-peptides and, therefore, a very high concentration of integrintargeting ligands. By mixing lipofectin with the oligolysine-peptide, then adding plasmid DNA or RNA complexes are formed containing all three components. The particles, due to the high density of ligands, have a high avidity for integrins on cell surfaces, bind and are internalised by a phagocytic process (Hart et al., 1994). The vesicles fuse to form endosomes where, under acid conditions, the DOPE element contained within the particles mediates destabilisation of the endosomal membrane and subsequent plasmid release into the cytoplasm. Phagocytosed particles lacking lipofectin are degraded in the endosomes. Particles lacking the integrin-targeting moiety are less efficient at cell binding and internalisation. Both lipofectin and the oligolysine ([K] ₁₆) element of the oligolysine-peptides probably contribute to the overall efficiency of the LID complexes but the integrin-targeting capacity of the oligolysine/peptide component appears to be important for optimal targeting and internalisation of the complexes.

Cystic fibrosis (CF) is the most common monogenic disorder in the Caucasian population. Morbidity is mainly associated with lung disease. CF is caused by mutations in the gene encoding the cystic fibrosis transmembrane conductance regulator protein (CFTR), a cell membrane channel that mediates secretion of chloride ions. Correction of this defect in the bronchial cells by CFTR gene transfer will correct the biochemical transport defect and, hence, the lung disease. Clinical trials so far have generated encouraging data but highlighted the need for more efficient, non-toxic vectors.

In accordance with the present invention integrin-targeted transfection vector complexes comprising peptide 6 above, which is an α5β1-targeted peptide, linked to oligolysine comprising 16 lysines i.e. K₁₆, (component I); lipofectin (component L); and a reporter gene (component D) were used with EGTA for transfection. Sub-confluent normal and CF epithelial cell lines were transfected with the green fluorescent report gene and transfection efficiencies of 40 to 50% were achieved. Cells were then cultured in confluent monolayers to model more closely airway structure. As expected, the transfection efficiency dropped dramatically for the confluent cells, being only about 5%. Co-treatment of confluent cells with EGTA restored the transfection efficiency to 20 to 30%. The same EGTA treatment of sub-confluent cells had no effect on transfection efficiency and there was no evidence that either treatment with EGTA or the transfection process itself increased the rate of mitosis in confluent monolayers. The vector, therefore, transfects confluent cells or other slowly dividing or non-dividing cells that are in contact with each other with much better efficiency when treated with EGTA. The treatment restores the transfection efficiency of confluent cells to about half of that seen in sub-confluent, rapidly dividing cells.

Further work showed that transfection efficiency of airway epithelial cells in vitro using both green fluorescent protein and luciferase reporter genes was increased about four-fold, and in vivo transfection of mouse lungs using luciferase reporter gene was also increased about four-fold. Inhibition of DNA polymerase and hence mitosis with aphidicolin showed that the increased transfection efficiency was not associated with any increase in mitosis.

As set out above, our observation that increased transfection efficiency results from the use of an agent that disrupts cell-cell junctions and is not associated with any increase in mitosis is surprising and is contrary to the accepted theory, which is that the low transfection efficiency observed with confluent cells treated with non-viral vectors is a function of the nuclear barrier present in such cells.

In cells that are dividing, the nucleus becomes accessible to plasmid DNA during mitosis an hence is not a barrier. Confluent cells are slowly dividing or non-dividing and hence substantially non-mitotic. The nucleus forms a barrier in such cells preventing access of plasmid DNA to the nuclear machinery. The use of an agent that disrupts cell-cell junctions between confluent cells increases the availability of receptors exposed on lateral membranes, which enhance binding and uptake of receptor-targeted vectors. If the nuclear barrier theory were correct, simply increasing the binding of vectors and hence uptake of DNA would not enhance transfection efficiency as the DNA would not be able to pass the nuclear barrier in the confluent cells. Our results indicate that mitosis is not increased by the treatment with is an agent that breaks down cell-cell junctions. Accordingly and contrary to accepted belief, the nucleus in the confluent cells is not acting as a transfection-limiting barrier. Furthermore, our finding that the calcium-binding agent EGTA enhances transfection efficiency is contrary to previous teachings that calcium ions are important for efficient transfection by polycationic complexes.

The following non-limiting Examples illustrate the present invention. Examples 1 to 14 relate to integrin-binding transfection vector complexes and their uses. Examples 15 and 16 illustrate the transfection of confluent cells in vitro and in vivo. The Examples refer to the accompanying drawings, in which:

### FIGURES

FIG. 1 shows the effect of different amounts of lipofectin (DOTMA:DOPE) on the enhancement of transfection of ECV304 cells using a complex consisting of lipofectin, oligolysine-peptide 1 ([K]₁₆GACRGDMFGCA [SEQ.ID.NO.: 22]) and plasmid pGL2.

FIG. 2 shows the effect of different amounts of lipofectin on the enhancement of transfection of A375M, COS-7 and ECV-40 cells using a complex consisting of lipofectin, oligolysine-peptide 1 ([K]₁₆GACRGDMFGCA) and plasmid pGL2.

FIG. 3 shows the effect of the order of mixing the components of a complex consisting of lipofectin (L), oligolysine-peptide 1 ([K]₁₆GACRGDMFGCA) (I) and plasmid pGL2 (D) on the enhancement of transfection of ECV40 cells.

FIG. 4 shows a comparison of enhancement of transfection by lipofectin of complexes containing plasmid pGL2 and oligolysine-peptide 1 ([K]₁₆GGACRGDMFGCA, pep 1), or oligolysine-peptide 5 ([K₁₆GACDCRGDCFCA [SEQ.ID.NO.: 22], pep 5), or oligolysine-peptide 6 ([K]₁₆GACRRETAWACG [SEQ.ID.NO.: 21], pep 6) or [K]₁₆ (K16), with lipofectin (lip) and without lipofectin, and a complex containing plasmid pGL2 with lipofectin: [K]₁₆lysine-peptide 1 in a ratio by weight of 4:1 (Lipo 4 to 1).

FIG. 5 shows the dose-dependency of a complex containing lipofectin, oligolysine-peptide 6 ([K]₁₆GACRRETAWACG) and plasmid pGL2 on the availability of integrin-binding ligands.

FIG. 6 shows the structure of various complexes, as determined using atomic force microscopy, the complexes being formed with different combinations of plasmid DNA (plasmid pGL2), oligolysine-peptide ([K]₁₆-peptide 6) and lipofectin as follows: A: [K]₁₆-peptide 6 and plasmid pGL2; B: [K]₁₆-peptide 6, lipofectin and plasmid pGL2; C: lipofectin and plasmid pGL2, optimal ratio; D: lipofectin and plasmid pGL2, suboptimal ratio.

FIG. 7 shows levels of expression of IL-12 48 hours after transfection of COS-7 cells and neuroblastoma cells lines IMR-32, KELLY and SHSY-5Y with a complex containing lipofectin, oligolysine-peptide 6 ([K]₁₆GACRRETAWACG) and either two retroviral plasmid constructs encoding the two domains of IL-12 (MFGS-IL12) or one plasmid containing a fusion gene, Flexi-12 under a CMV promoter.

FIG. 8 shows the effect of transfection with anti-sense oligonucleotides (AS) to the thrombin receptor (PAR-1) on thrombin induced proliferation of human foetal lung fibroblasts (HFL-1 cells).

FIG. 9 shows the effect of transfection of haematopoietic cell lines HL60, PLB985, TF1 and U937 with LID complexes containing lipofectin, the reporter gene pEGFP-N1 and either [K]₁₆-peptide 6 (pep 6) or [K]₁₆-peptide 8 (GGCRGDMFGCA [SEQ.ID.NO.: 8] pep 8) compared with untreated cells. The percentage of GFP positive cells is determined using a fluorescence activated cell sorter.

FIG. 10 shows GFP (green fluorescent protein) transfected confluent human airway epithelium cells. FIG. 10A shows the cells transfected by a transfection vector complex in the absence of EGTA, FIG. 10B shows transfection in the presence of EGTA.

FIG. 11 shows the effect of EGTA on transfection of mouse lung in vivo using luciferase as the reporter gene. Results are given in RLU per mg lung protein for transfection with no EGTA, with 100 mM EGTA and with 400 mM EGTA.

FIG. 12 shows flow cytometry analysis on the transfection efficiency with pEGFP in subconfluent (A, B) and confluent (C, D) 1HAEo-cells (A, C) and 2CFSMEo-cells (B, D). High efficiency was observed in subconfluent cells but it dropped dramatically when the cells became confluent.

FIG. 13 shows luminometric assay of the effects of EGTA (A, B) and anti-E-cadherin (C, D) on transfection. The confluent (A) and subconfluent (B) 1HAEo-cells were transfected with LID complexes after pre-treatment with EGTA (EGTA) or in the presence of EGTA (LID+EGTA). Significant enhancement effects were observed in confluent but not subconfluent cells with LID vector incorporated with EGTA (LID+EGTA) compared with either the EGTA pretreatment (EGTA) or the control transfection without EGTA (OptiMEM). Significant increase in transgene luciferase activities was also observed in confluent 2CFSMEo-cells (C) incubated with anti-E-Cadherin in three different concentrations compared to the transfection without antibody (labelled as LID) (p<0.05) or in the presence of IgG controls (labelled as LID+IgG control). Again, no significant difference was observed in sub-confluent cells (D) transfected in the presence of either anti-E-Cadherin or IgG control.

FIG. 14 shows flow cytometry analysis of EGTA effects on transfection efficiency (A-C) and the rate of BrdU incorporation (D-F) in confluent 1HAEo-cells. Cells were. transfected with pEGFP (A) or pEGFP plus EGTA (B), or pEGFP plus EGTA and aphidicolin (C). In parallel with the transfection, cells were labelled with BrdU (D) or BrdU plus EGTA (E), or BrdU plus EGTA and aphidicolin (F). 8.6% cells expressed transgene EGFP (A). EGTA increased the efficiency to 31.7% (B) and this enhancement effect was still observed in the presence aphidicolin where the EGFP-positive cells accounted for 23.6% (C). Meanwhile, EGTA showed no effects on cell proliferation with the rate of BrdU incorporation being 7.1% in confluent cells (D), 6.5% in the presence of EGTA (E), and 5.3% in the presence of EGTA and aphidicolin (F).

FIG. 15 shows sub-confluent 1HAEo-cells: flow cytometry analysis of EGTA effects on transfection efficiency (A-C) and the rate of BrdU incorporation (D-F). Cells were treated in the same way as in the confluent cells shown in FIG. 13.

Transfection efficiency was as high as 44.5% in sub-confluent cells (A). Similar efficiency of 45.6% was observed in the presence of EGTA (B) and there were still 24.8% cells being transfected in the presence of aphidicolin (C). Instead of promoting proliferation, EGTA decreased BrdU incorporation rate in sub-confluent cells as the proportion of BrdU-positive cells dropped from 43.0% (D) to about 23.9% after EGTA treatment (E). 7.8% cells were labelled with BrdU in the presence of aphidicolin (F).

FIG. 16 shows double immunofluorescence for transgene β-Galactosidase (transgene expression shown by green cytoplasmic staining, irregular shapes) and BrdU labelling (shown by red nuclear staining, smaller rounder shapes) in subconfluent (A) and confluent (B, C) 1HAEo-cells. Most of the subconfluent cells were labelled with BrdU and many of them expressed transgene simultaneously (irregular shape containing rounded shape), shown in FIG. 15A. Only few confluent cells were positive for β-Galactosidase while few were labelled with BrdU (B). After EGTA treatment many more cells expressed transgene see FIG. 15C while their proliferation state was not affected as evident by the unchanged rate of BrdU labelling, few cells were stained red.

FIG. 17 shows photomicrographs of confluent 1HAEo-cells showing the effects of EGTA treatment. The images in left-hand column (A, C, E, G) shows the cells before EGTA treatment and the right-hand column (B, D, F, H) are the cells after EGTA treatment. They are representative images of the typical morphology of live confluent cells (A, B: ×400, conventional inverted microscope), immunofluorescence for tight junction protein occludin (C, D: ×400 projected images, confocal microscope), binding of LID complexes to cell surfaces (E, F: ×100, inverted confocal microscope) and the expression of transgene PEGFP (G, H: ×100, conventional inverted microscope Confluent cells form an un-permeable cell sheet (A). Tight junctions were clearly confined around cells as showed by immunofluorescence for occludin (C). This allowed only a few particles of LID vectors to bind on to the cell surface (E) and only a small number of cells expressed transgene EGDP (G). After treatment with EGTA, the confluent cells rounded up (B). Tight junctions were disrupted as showed by the broken lines of immunofluorescence for occludin (D) and, much more LID complex particles bound to the cell surfaces (F) and consequently, more cells expressed transfene EGFP (H).

The following non-limiting Examples illustrate the invention. The Materials and Methods section relates to all the Examples, unless specified otherwise. Examples 1 to 12 illustrate the production of transfection complexes and, as comparative examples, illustrate the transfection of cells that are dividing. Examples 13 to 15 illustrate the transfection of non-dividing and/or confluent cells.

### EXAMPLES

### Materials & Methods

Cell Lines

The cell line COS-7 (monkey kidney epithelial cells) were maintained in Dulbecco's Modified Eagle Medium (DMEM; Life Technologies, Paisley, U.K.) supplemented with 10% foetal calf serum (FCS), L-glutamine, penicillin and streptomycin. ECV304 (spontaneously transformed human umbilical vein endothelial cells) were grown in 199 Medium (Life Technologies, Paisley, U.K.). HT1080 fibrosarcoma cells and A375M melanoma cells were maintained in DMEM and 10% FCS. IMR2 neuroblastoma cells were grown in DMEM F12 Nutrient Mix (Life technologies). Cell lines were all grown in a 37° C. incubator with a 5% CO₂ water-saturated atmosphere.

Peptide Synthesis

The sequence of peptide 6, GACRRETAWACG, was based on an α5β1-specific peptide from a phage display library (Koivunen et al., 1995). The oligolysine-peptide [K]₁₆GACRRETAWACG was synthesised as follows:

Protected amino acids and preloaded Gly-Wang resin were obtained from Calbiochem-Novabiochem (Nottingham, U.K.). Solvents and HBTU [2-(1H-benzotriazol-1-yl)-1,1,3,3-tetram-ethyluronium hexafluorophosphate] were obtained from Perkin-Elmer Applied Biosystems, U.K. The peptide was synthesised on a Model 431A updated Applied Biosystems Solid Phase Synthesiser on 0.1 mmol preloaded Gly-Wang resin (Calbiochem-Novabiochem, Nottingham, U.K.) using basic feedback monitoring cycles and HBTU as a coupling reagent. 9-fluorenylmethyloxy-carbonyl was used for temporary α-amino group protection. Side-chain protecting groups were tert-butyloxycarbonyl for Lys and Trp, trityl for Cys, 2,2,5,7,8-pentamethylchroman-6sulphonyl for Arg, tert-butylester for Glu and tert-butyl ether for Thr. Cleavage from the resin and deprotection of the peptide was achieved by treating the peptidyl-resin with 10 ml of a mixture containing 10 ml trifluoroacetic acid, 0.25 ml ethanedithiol, 0.25 ml triisopropylsilane at 20° C. for two hours. The peptide was precipitated using ice-cold diethyl-ether and then filtered through a fine sintered glass filter funnel under light vacuum. The peptide precipitate was dissolved in 10% acetic acid/water solution and freeze dried. The crude peptide was analysed by reverse phase HPLC and matrix assisted laser desorption ionisation time of flight mass spectroscopy. Purity of the crude peptide was about 70% by reverse phase HPLC, and mass analysis using a Finnegan LazerMat gave a molecular weight of 3331.5 for the MH+ ion which was in excellent agreement with calculated weight for MH+ ion of 3331.46.

Oligolysine-peptide 1: (K]₁₆GACRGDMFGCA and oligolysine-peptide 5: [K]₁₆GACDCRGDCFCA were obtained from Zinsser Analytic (Maidenhead, U.K.).

Plasmid DNA

The plasmids pGL2, which contains a luciferase reporter gene (Promega, Madison, Wis., U.S.A.) and pCMVβ, which contains a β-galactosidase reporter gene (Clontech, Palo Alto, Calif., U.S.A.) were grown in Escherichia coli DH5α and purified, after bacterial alkaline lysis, on Qiagen resin columns (Qiagen Ltd., Crawley, U.K.) by the manufacturer's instructions. Isopropanol-precipitated DNA pellets were washed with 70% ethanol then dissolved in water or TE buffer (10 mM Tris-Cl, pH 8.0 and 1 mM EDTA).

Spectrophotometric measurements of plasmid solutions were used to assess plasmid concentration (A₂₆₀) and purity (A₂₆₀/A₂₈₀ ratio). Plasmid solutions were adjusted to a concentration of 1 mg/ml and stored at 4° C.

Formation of Transfection Complexes

Cells were seeded into 24-well plates at 5×10⁴ cells per well then incubated overnight at 37° C. in complete growth medium. The following day, transfection complexes were made from the following stock solutions, all prepared in OptiMEM (Life Technologies, Paisley, U. K.), lipofectin (an equimolar mixture of the cationic lipid N-[1-(2,3-dioleyloxy)propyl]-N,N,N-trimethylammonium chloride (DOTMA) and the neutral lipid dioleoyl phosphatidylethanolamine (DOPE), obtained as “Lipofectin” from Life Technologies, Paisley, U.K.) (1 mg/ml), pGL2-control (1 mg/100 ml) and [K]₁₆]/integrin-targeting peptide 1, 5 or 6 (0.1 mg/ml).

Complexes were made usually with three components: oligolysine-peptide (I), plasmid DNA or RNA (D) and lipofectin (L), by mixing together the different components with an automatic pipette. The mixture LID was made in the same way in the optimal weight ratio 0.75:4:1 (L:I:D). Both types of mixture were left to aggregate for at least 30 min then diluted to a concentration of one microgram DNA per 0.5 ml with OptiMEM. The growth medium was removed from each well then 0.5 ml of transfection complex added. The plate was then returned to the incubator for four to six hours. The transfection medium was then removed and replaced with 1 ml of complete growth medium. Transfected cells were incubated for 48 to 72 hours then assayed for reporter gene activity.

Luciferase Assays

Cells transformed with pGL2 were washed twice with PBS to remove serum then 100 microlitres of Reporter Lysis Buffer (Promega, Madison, Wis, U.S.A.) was added to each well and placed at 40° C. for 15 to 30 minutes. Cells were then dislodged by scraping with a yellow micropipette tip. Cell-free lysates were then prepared and assayed with a Luciferase Assay kit (Promega, Madison, Wis., U.S.A.) following the manufacturer's instructions. Total light emission was measured for 60 seconds on an LKB 1251 Luminometer (Labtech, Uckfield, U.K.). The protein concentration of each sample was then determined with Protein Assay Reagent (BioRad, Hercules, Calif., U.S.A.) and luciferase enzyme activity expressed in terms of relative light units per milligram of protein (RLU/mg).

LacZ Assays

β-galactosidase activity was detected by staining with X-gal. After washing with PBS cells were fixed to the plastic plates by addition of 0.5% glutaraldehyde in PBS for 20 minutes at 40° C. Wells were washed with PBS and cells were stained with X-gal at 370 C for up to six hours.

Atomic Forces Microscopy (AFM)

Atomic forces microscope analysis of transfection complexes was performed as described previously (Wolfert & Seymour, 1996) using an AFM-2, part of the NanoScope II (Digital Instruments, Santa, Barbara, U.S.A.). Transfection complexes of [K]₁₆-peptide 6/pGL2, with and without lipofectin, were prepared as described above except that water was used as the diluent for all components rather than OptiMEM.

### Example 1

### Effect of Different Amounts of Lipofectin (DOTMA/DOPE) on Transfection

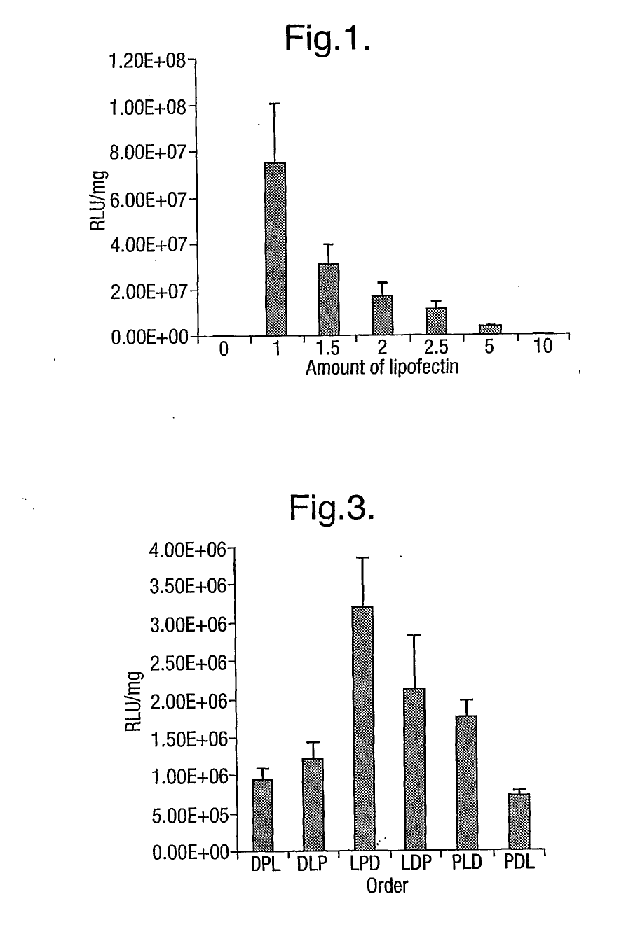

Transfection complexes were prepared as described above in the Materials & Methods section. The complexes were made by mixing solutions of oligolysine-peptide 1 ([K]₁₆GACRGDMFGCA) at 0.1 mg/ml in OptiMEM low serum tissue culture medium with a solution of lipofectin (DOTMA/DOPE cationic liposome as above) in a range of concentrations from 1 to 10 μg/100 μl in OptiMEM. Finally, the appropriate amount of pGL2-control plasmid DNA (0.1 mg/ml) was added and mixed by repeated pipetting. The ratio of mixing of each component was a constant 4 μg of oligolysine-peptide per μg of DNA, while the proportion of lipofectin varied from 1 to 10 μg per μg of DNA. ECV304 cells were transfected with the complexes as described above, incubated for 48 hours then assayed for luciferase expression as described above. The results are shown in FIG. 1.

Complexes formed with 1 μg of lipofectin and 4 μg of oligolysine-peptide per microgram of plasmid were almost 100-fold more active than complexes lacking lipofectin. Addition of larger amounts of lipofectin reduced transfection activity in a lipofectin dose-dependent manner.

Similar results were obtained with [K]₁₆-peptide 6.

### Example 2

### Effect of Different Amounts of Lipofectin on Transformation in Three Different Cell Lines

Experiments were then performed to refine the optimal amount of lipofectin in LID transfection complexes using three different cell lines A375M (melanoma cells), COS-7 (monkey kidney epithelial cells) and ECV304 (human umbilical cord endothelial cells).

Transfection complexes were made as described in Example 1 but using a narrower range of amounts of lipofectin. Lipofectin/oligolysine-peptide/DNA complexes were prepared with constant amounts of [K]₁₆-peptide 1 ([K]₁₆GACRGDMFGCA) (4 μg) and pGL2 (1 μg) plasmid DNA and a range of lipofectin amounts (1 to 2.5 micrograms). Complexes were used to transfect A375M, COS-7 and ECV304 cells, which were then harvested two days later for luciferase expression analysis.

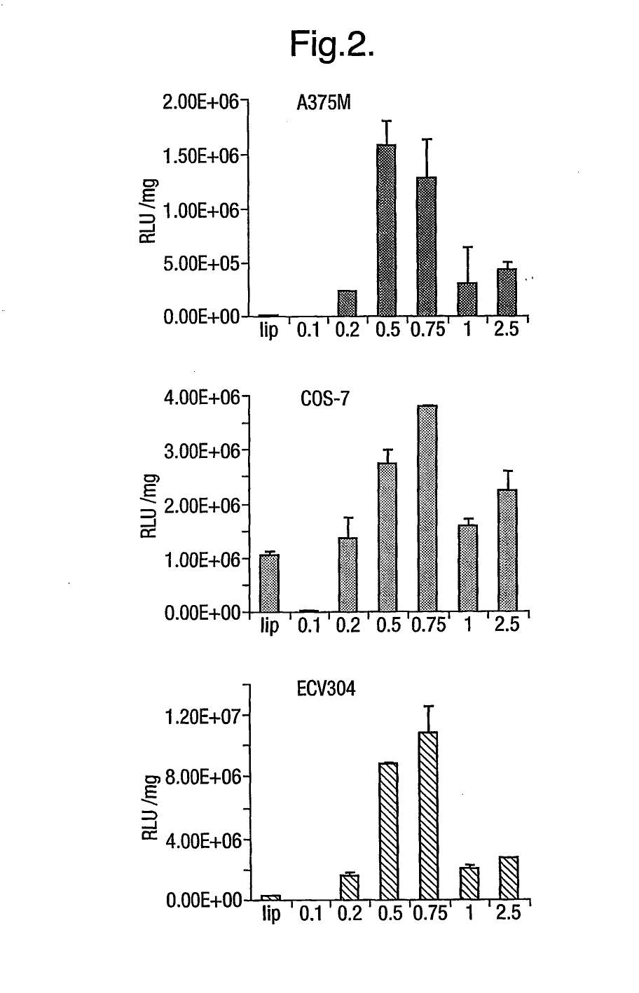

The results are shown in FIG. 2. In each case the optimal transfection ratio peaked at 0.75 μg of lipofectin per microgram of plasmid DNA. This combination of the amounts of the components was maintained in all subsequent examples.

A mixing ratio L:I:D of 0.75:4:1 by weight corresponds to a molar ratio of 0.5 nmol lipofectin: 1.25 nmol oligolysine-peptide 1: 0.25 pmol pGL2-control. The molar charge of each component is 0.5 moles positive charge per mole lipofectin, seventeen moles positive charge per mole [K]₁₆-peptide 1 and 12,000 moles negative charge per mole of pGL2 (6 kb). There-fore, in the optimal transfection complex, 3 nmol of negative charge from the plasmid is mixed with 21 nmol of positive charge from oligolysine-peptide 1 and 0.25 nmol positive charge from lipofectin. Hence the charge ratio of approximately 7:1 positive to negative charges in ID complexes is little altered by the incorporation of 0.25 nmol positive charge from lipofectin into high efficiency LID transfection complexes. It is likely, therefore, that the improvement in transfection efficiency of LID complexes is not charge related.

### Example 3

### Effect of the Order in Which the Components of the Complex are Mixed

To determine the procedure for the production of optimal LID transfection complexes transfections were performed with complexes made by adding the components of the complexes in different orders. All combinations were prepared with the same amounts and concentrations of the components (1 μg pGL2 plasmid DNA, 0.75 μg of lipofectin and 4 μg of oligolysine-peptide 1 ([K]₁₆GACRGDMFGCA). Transfections were performed in ECV304 cells and luciferase activity was assessed as described above.

The results are shown in FIG. 3 in which D represents the plasmid vector pGL2, I represents [K]₁₆-peptide 1 and L represents lipofectin. The expression data indicates that the order of mixing LID was optimal. Significantly, combinations in which the lipofectin was the last component added were least efficient. The order of mixing, LID, was employed in all subsequent transfection experiments.

### Example 4

### Transfection Rates

Cells were transfected with optimised oligolysine-peptide/lipofectin/pCMVβ complexes as described in Examples 1 and 2 prepared in the order of mixing LID but using pCMVβ as the plasmid vector (component D) instead of pGL2. The cells were stained for β-galactosidase activity with X-gal as described above. A number of cell types, A375M, COS-7 and ECV304 displayed transfection efficiencies of 50 to 100% compared to 1 to 10% achieved with oligolysine-peptide/DNA complexes alone. This represents a very significant improvement in transfection efficiency.

### Example 5

### Comparison of Enhancement with Lipofectin and with Different Oligolysine-peptides

To compare the effect of different integrin-targeting oligolysine-peptides, duplicate sets of complexes were formed with plasmid pGL2 and one of the following: oligolysine-peptide 1 ([K]₁₆GACRGDMFGCA, pep 1), oligolysine-peptide 5 ([K]₁₆GACDCRGDCFCA, pep 5), oligolysine-peptide 6 ([K]₁₆GACRRETAWACG, pep 6), and [K]₁₆. One set of complexes also contained lipofectin (lip), the other was without lipofectin. A control complex containing plasmid pGL2 with lipofectin and [K]₁₆lysine-peptide 1 in a ratio by weight of 4:1 was prepared.

Each complex was used to transfect cell lines and luciferase expression determined. Complexes were made with (lip) and without lipofectin. An optimised complex was performed for comparison. All oligolysine-peptides were mixed with lipofectin and plasmid DNA (KLD) in the same optimised charge ratios and order of mixing.

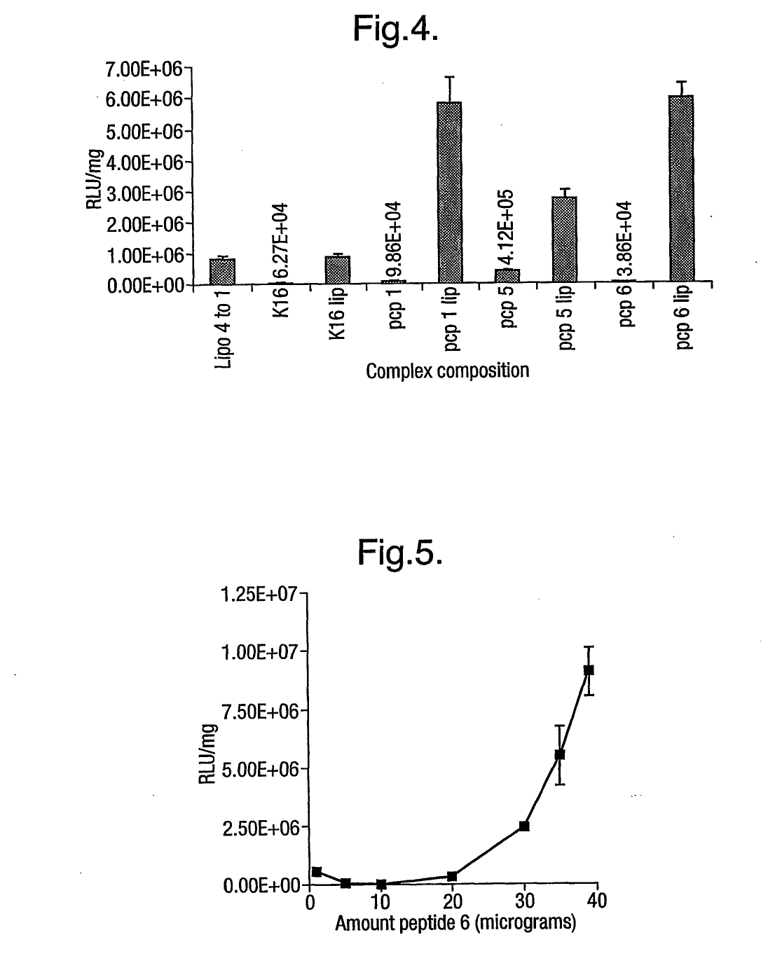

The results are shown in FIG. 4. Although KLD complexes were usually better transfection agents than KD or LD complexes, LID complexes generated luciferase expression levels three to six-fold higher than KLD complexes. Expression levels from LID complexes containing oligolysine-peptide 5 were two-fold lower than those containing oligolysine-peptide 1 or oligolysinepeptide 6, which may reflect the differing integrin receptor affinities of the peptides. The transfection enhancement of the LID complexes was observed with all the peptides tested, two of which (peptides 1 and 5) contain the conserved RGD sequence, one of which (peptide 6) does not.

### Example 6

### Specificity

To demonstrate integrin specificity, LID complexes were prepared with constant amounts of plasmid pGL2-control and lipofectin, and a range of combinations of [K]₁₆-peptide 6 and [K]₁₆. A total of 40 μg of [K]₁₆-peptide was used, consisting of 1, 5, 10, 20, 35, 39 μg of [K]₁₆-peptide 6 made up to 40 μg with [K]₁₆.

Transfections were performed as described in Example 1 and luciferase assays performed after 48 hours. The results are shown in FIG. 5. Transfection efficiency demonstrated an apparently exponential increase with increasing amounts of oligolysine-peptide 6, and, therefore, a dose-dependent response to the amount of available integrin-binding ligands. Accordingly, while both the sixteen-lysine domain, and the lipofectin components are themselves capable of mediating transfection, both individually and in [K]₁₆/lipofectin combination complexes, the highest efficiency transfection is directly proportional to the amount of available integrin-binding ligand.

### Example 7

### Atomic Force Microscopy

Atomic force microscopy experiments were performed to determine and compare the structures formed by mixing 4 μg [K]₁₆peptide 6 and 1 μg pGL2-control plasmid DNA (ID complexes). LID complexes were formed from [K]₁₆-peptide 6 (4 μg)/lipofectin (0.75 μg)/DNA (1 μg) in the order LID which was shown to yield optimal transfection results.

Lipofectin/DNA complexes (LD) were formed at two different ratios; an optimal transfection ratio of 5 μg lipofectin per microgram of pGL2 and the same ratio as used in LID complexes, 0.75 μg lipofectin per microgram of plasmid.

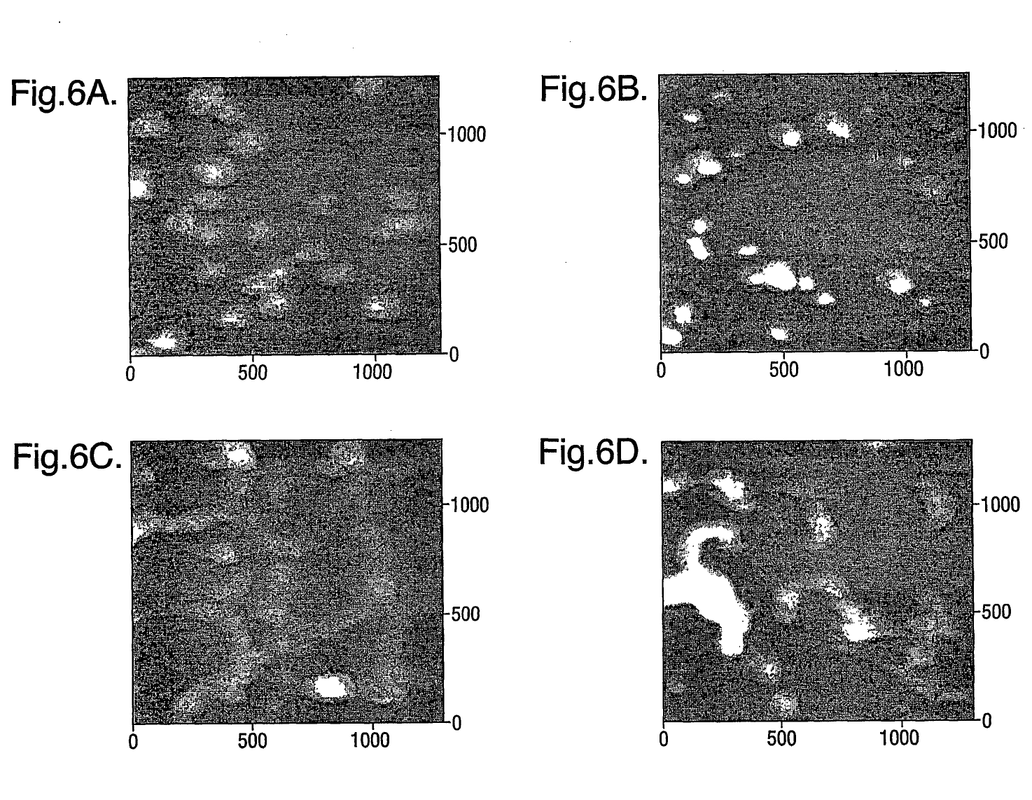

The results are shown in FIG. 6. ID complexes, composed of oligolysine-peptide 6 and plasmid DNA, were examined initially by AFM within fifteen minutes of mixing the two components. The complexes formed particles of low poly-dispersity which, on the mica coverslips, had a diameter of approximately 200 nm. A computer-generated contour map revealed that the particles formed were of irregular conical shape. LID complexes assessed by AFM formed particles of a similar size and shape to ID complexes. The additional lipofectin did not, apparently, disrupt the particles. LD complexes, however, formed at the 5:1 ratio appeared as a network of tubes with occasional particles associated with the tubes. LD complexes formed at the lower ratio (0.75:1), however, appeared to be short tubular structures. LD complexes formed at this lower ratio were inactive in transfection experiments.

LID complexes formed as above were also analysed by AFM after standing overnight. Particles were now smaller in size with diameters of approximately 50-100 nm suggesting that the particles had compacted. Computer-generated computer maps represented these particles as regular conical structures. The cones were measured and their volumes were calculated. The spheres which the particles are predicted to form when free in solution were then calculated to be 20 to 60 nm in diameter. In transfection experiments with pGL2 the compact particles formed overnight in water yielded luciferase expression results approximately twice as high as the freshly made complexes.

### Example 8

### Transfection of Neuroblastoma Cells

Transfection of three different human neuroblastoma cell lines, SHSY-5Y, KELLY and IMR-32 and one mouse neuroblastoma cell line, Nb2A, was optimised using an LID complex containing [K]₁₆-peptide 6, lipofectin and either luciferase or GFP as reporter gene, as described in the Materials and Methods section and the Examples above.

The three human neuroblastoma cell lines and COS-7 cells were then transfected using the same LID complex with, instead of the reporter gene, one of two different IL-12 expressing vectors. One vector expresses a fusion protein of the two chains of IL-12, p35 and p40, (Flexi-12; Anderson et al. 1997) This fusion is regulated by a CMV promoter. The second IL-12 expression system consists of two retroviral constructs MFGS-p35 and MFGS-p40, which are retroviral plasmid constructs encoding the two separate chains of interleukin-12 (IL-12). Both genes are regulated by the retroviral long terminal repeats (LTRs). The vectors were obtained from Professor Mary Collins, UCL, London.

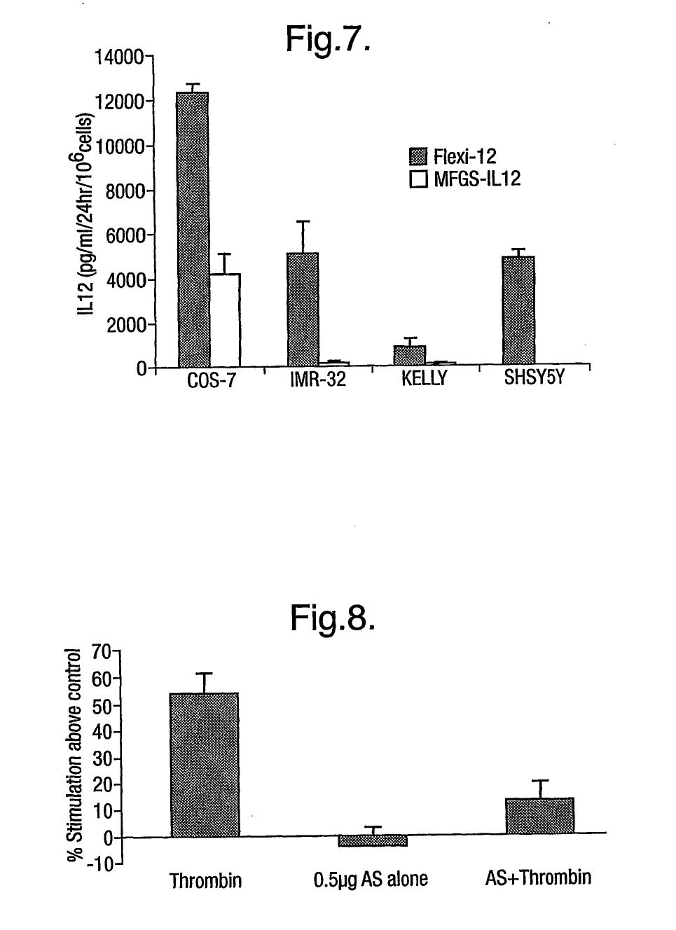

Secreted IL-12 expression was monitored by ELISA 48 hours after transfection. The transfected cells were found to secrete high levels of the cytokine, see FIG. 7 The Flexi-12 construct was most efficient.

These results demonstrate that the transfection system of the present invention is suitable for use in a vaccine for neuroblastoma, an important childhood malignancy, and also for vaccines against other cancers.

### Example 9

### Transfections of Primary Smooth Muscle Cells and Cardiac Myocytes

Tissue cultures of rat primary smooth muscle cells (aortic smooth muscle cells) and cardiac myocytes were prepared according to standard methods (Blank et al. 1988). An LID complex comprising lipofectin, [K]₁₆-peptide 6 and GFP as a reporter gene in the optimal LID ratio and mixing order was prepared as described in the Materials and Methods section and the Examples above. The tissue cultures were transfected with the LID complex as described in the Material and Methods section above. Fluorescing imaging of GFP-expressing cells demonstrates transfection efficiency in excess of 50%.

Primary smooth muscle cells and cardiac myocytes are particularly resistant to plasmid-mediated transfection using most other non-viral vectors. In contrast, the transfection complex of the present invention achieved transfection efficiencies in excess of 50%, thus demonstrating the utility of the complexes for treatment of diseases affecting muscle, including smooth muscle and cardiac muscle.

### Example 10

### Transfections with High Molecular Weight Constructs

Different size constructs can be delivered with the transfection complex of the present invention. A fibroblast culture was transfected as described in the Materials and Methods section with an LID complex comprising [K]₁₆-peptide 6, lipofectin and a 130 kB DNA construct. The complex, comprising the LID components in the optimal ratio and mixing order, was prepared as described in the Methods and Materials section and Examples above. Transfection was achieved with 2-3% efficiency.

Cellular process associated with the enhanced integrin-mediated internalisation of DNA using a complex of the present invention are more closely related to phagocytosis than endocytosis and are thus particularly suited to the delivery of complexes containing very large DNA molecules.

### Example 11

### Transfection With Anti-sense DNA

Thrombin stimulates proliferation of human lung fibroblasts. Thrombin-treated human lung fibroblasts (HFL-1 cells) proliferated 53% in response to thrombin. 24 hours before treatment with thrombin, HFL-1 cells were treated with an LID complex comprising [K]₁₆-peptide 6, lipofectin and a 20-mer antisense oligonucleotide directed against the thrombin receptor PAR-1 in the optimal ratio and mixing order prepared as described in the Materials and Methods section and the Examples above. The antisense oligonucleotide-containing complex was in contact with the cells for 4 hours. 24 hours after the start of the treatment with the complex, treatment with thrombin was carried out.

The thrombin-induced proliferation was attenuated by 76% +/− 12% by the pre-treatment with the LID complex. Cells treated with the antisense-containing complex but not with thrombin did not proliferate, see FIG. 8.

This result demonstrates the utility of the complex of the invention for efficient intracellular transport of antisense oligonucleotides, as is required for antisense therapy, for example, antiviral and anticancer therapy.

### Example 12

### Transfection of Haematopoietic Cells

Haematopoietic cells are particularly resistant to transfection with most plasmid-mediated vectors.

LID complexes were prepared as described in the Material and Methods section and Examples above using lipofectin and [K]₁₆-peptide 6, which targets α5β1 integrins, and pEGFP-N1 (Promega) as reporter gene. Complexes were prepared analogously substituting [K]₁₆-peptide 8 ([K]₁₆GACQIDSPCA SEQ.ID.NO.: 21), which targets α4β1 integrins, for [K]₁₆-peptide 6. The complexes were prepared by mixing the components in the optimal ratio and mixing order as described in the Materials and Methods section and Examples above.

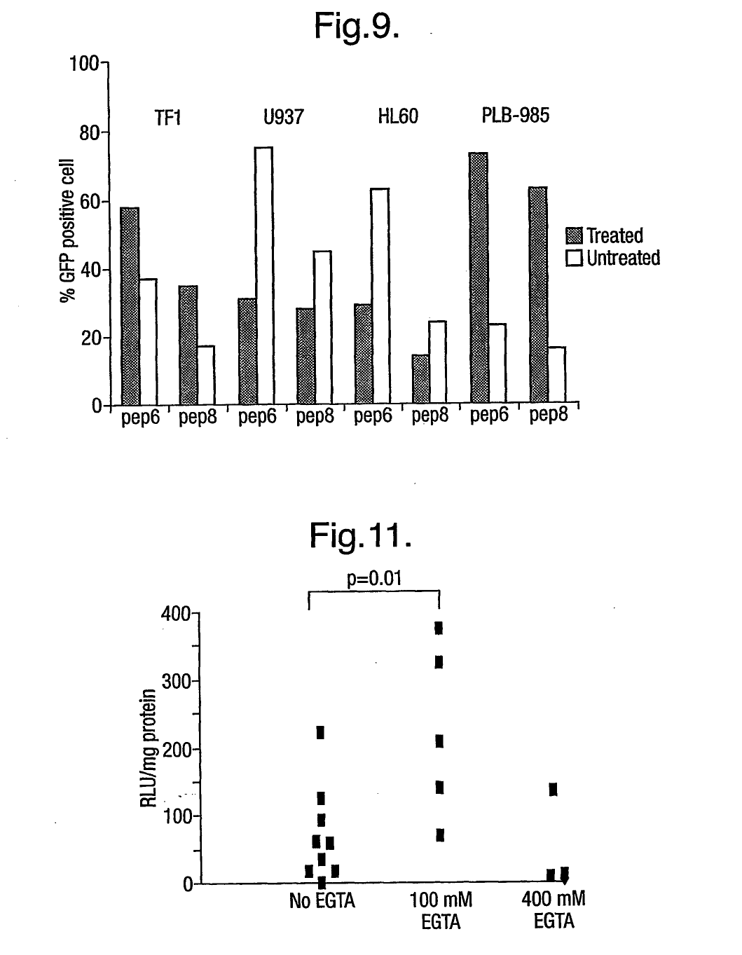

Four different haematopoietic cells lines (HL60, PLB985, TF1 and U937) were transfected as described in the Materials and Methods section with the following modifications: cells were untreated or were treated with GM-CSF (10 ng/ml) for TF1 cells or phorbol myristic acid (PMA) for the other three cells lines prior to transfection. Transfection with the LID complexes containing pEGFP-N1 generated a transfection efficiency of more than 60% in all four lines as measured on fluorescent activated cell sorter, see FIG. 9.

These results demonstrate the utility of the transfection complex of the invention for gene therapy involving haematopoietic cells, for example, gene therapy of leukaemia and bone marrow stem cell disorders. This is particularly useful because, as pointed out above, haematopoietic cells are particularly resistant to transfection with most plasmid-mediated vectors.

### Example 13

### Transfection of Confluent Human Airway Epithelial Cell in Vitro in the Presence of EGTA

Cell Cultures

Human airway epithelial (HAE) cells, type HEAO— were seeded at 80% into 24 well plates. The cells were grown in Modified Eagle Medium (MEM) obtained from Life Technologies (Paisley, Scotland) supplemented with 10% foetal calf serum and 2 mM L-glutamine to form cultures of confluent cells. Subconfluent cultures of the same cells were also prepared.

Reporter Genes

The reporter genes used were a green fluorescent protein (GFP) reporter gene and a luciferase reporter gene. The GFP reporter gene was used in the form of plasmid pEGFP-N1, obtained from Clontech. The luciferase reporter gene was used in the form of construct pCILuc consisting of the firefly luciferase gene (Luc) inserted into the expresssion vector pCI (Promega, Southampton, England)

Peptide

For these in vitro experiments peptide 12 [K]₁₆XSXGACRRETAWACG (X: ε amino hexanoic acid), SEQ.ID.NO.: 26, was used. The oligolysine-peptide component I comprised the integrin-targeted peptide CRRETAWACG, which targets the integrin α5 receptor, with a spacer XSXGA (X: ε amino hexanoic acid), and the oligolysine [K]₁₆, giving the sequence [K]₁₆XSXGACRRETAWACG (Zinsser Analytic, Maidenhead, UK).

Transfection Vector/EGTA Complexes

Transfection vector complexes were prepared as described in the Material and Methods section, see also Example 15. The complexes were prepared using 4 μg of the peptide, 0.75 μg of lipofectin and 1 μg of the reporter gene. All components and the vector complex were made up in OptiMEM (Life Technologies, Inc.). EGTA (Sigma) at a concentration of 100 mM in water was added to the transfection vector complex preparation at one hundredth of the volume of the vector complex, giving a final EGTA concentration of 1 mM.

Transfection

The EGTA/vector complex mixture was added to the cultures, which were then harvested two days later. As controls, cultures were treated with OptiMEM medium alone and with the transfection vector complex without EGTA.

To investigate whether mitosis affected the results, is transfection was carried out in the presence of aphidicolin, which inhibits DNA polymerase-α, which prevents transition of the cells from G1 to S phase. To arrest cell division Aphidicolin (Sigma, Poole, Dorset, England), was added to the cell cultures at a concentration in the range of 1 to 20 μg/ml 24 hours prior to transfection and was maintained throughout the rest of the experiment until the cells were harvested.

Transfection levels using the luciferase reporter gene was measured as described in the Material and methods section. In the case of the GFP reporter gene, after harvesting the cells were analysed by fluorescence activated cell sorting (FACS) for quantification of the number of fluorescent cells.

The results obtained using the GFP reporter gene are presented in Table 2 below:

TABLE 2

| Transfection agent(s) | Sub-confluent cells | Confluent cells |
|---|---|---|
| Control (no vector) | 0.3% | 0.7% |
| Vector, no added EGTA | 44.6% | 8.9% |
| Vector plus EGTA | 45.7% | 32.4% |
| Vector, EGTA, aphidicolin | 24.8% | 24.4% |

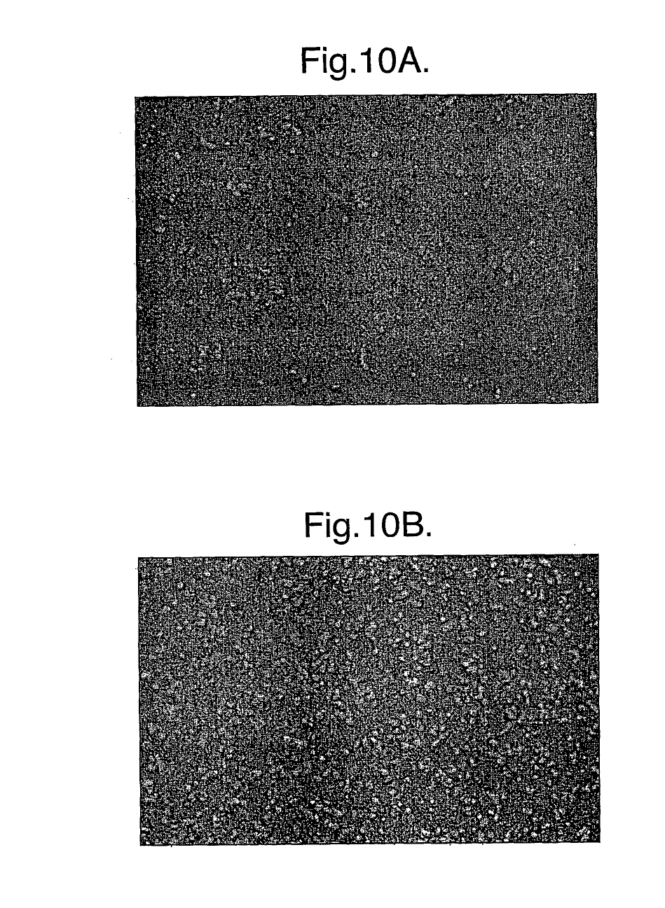

FIG. 10 shows clearly the influence of EGTA in the increase in the number of fluorescent cells transfected with the vector in the presence of EGTA (FIG 10B) compared with the vector alone, as shown in (FIG. 10A).

These results show that while the transfection vector complex transfects sub-confluent cells effectively, the transfection efficiency of confluent cells is very low. However, the presence of EGTA increases the transfection levels of the confluent cells four-fold, while having no effect on the efficiency of transfection of sub-confluent cells.

Bromodeoxyuridine (BrdU) labelling enables assessment of cell proliferation. BrdU kits are available from Zymed Laboratories, Inc., South San Francisco, Calif., U.S.A. BrdU was added to the cells at the same time as the transfection reagent and was maintained throughout the rest of the experiment until cell harvesting. The results of BrdU labelling of sub-confluent and confluent HAE cells is given in Table 3 below.

TABLE 3

| | Sub-confluent cells | Confluent cells |
|---|---|---|
| Control | 0.7% | 0.5% |
| BrdU | 44.4% | 7.1% |
| BrdU plus EGTA | 25.1% | 6.5% |
| BrdU plus aphidicolin | 1.3% | 5.3% |

These results, and those shown in Table 2 for transfection in the presence of aphidicolin, demonstrate that neither the treatment with EGTA nor the transfection process itself increases the rate of mitosis in the confluent HAE cells.

We obtained essentially the same results using the luciferase reporter gene instead of GFP. Transfections ere carried out using the transfection vector prepared as described above with the construct pCILuc, with and without EGTA, as described above. Luciferase assays were performed on cell-free extracts of harvested cells using a kit obtained from Promega using an Anthos Lucy 1 Luminometer. As with the GFP experiments, luciferase levels were enhanced approximately four-fold in confluent cells treated with EGTA compared with transfections performed without EGTA.

The finding that the presence of EGTA enhances transfection efficiency in the absence of mitosis is contrary to previous teachings, that mitosis is necessary for effective transfection.

Previous reports of in vitro transfection using a viral vector and EGTA showed that pre-incubation with EGTA (for 20 minutes) was required before the addition of vector (Wang, 1998). We found that pre-incubation of the cells with EGTA before transfection with the integrin-targeted transfection vector complex was no better than the results without EGTA.

We found the best results were when the EGTA and the vector were admixed before application to the cells.

A further difference from previously reported results (Wang, 1998) is that Wang described the use of EGTA with hypotonic solutions such as water, which are themselves known to promote the disruption of tight cell junctions. We found the use of the isotonic buffer OptiMEM to be successful.

We also observed enhancement of transfection in vitro in confluent HAE cells when anti-cadherin IgG was used instead of EGTA with the transfection vector complex described above. The anti-cadherin IgG was obtained from Serotec and applied at 50 μg/ml one hour before addition of the vector. This led to a 5 to 10 fold enhancement of luciferase activity in confluent cells whereas subconfluent cells showed no enhancement. These results confirm our theory that the enhancement results from increased availability of receptors and that, surprisingly, cell division is not required.

### Example 14

### Transfection of Mouse Lungs in Vivo in the Presence of EGTA

The protocol is different from that for the in vitro experiments described in Example 13, because of the importance of minimising the final volume of the vector to be administered.

Lipid (lipofectin) (1 mg/ml) and peptide 6 (GACRRETAWACG) (1 mg/ml) were mixed in one tube and pCILuc Plasmid DNA (1 mg/ml) and EGTA in phosphate buffer saline (PBS) were mixed together in another tube then the contents of the two tubes were mixed so that the final ratios were 0.75 μg lipid to 1 μg DNA and a final concentration of 100 mM EGTA (or no EGTA for the control). The protocol was repeated to give vector complexes having a final EGTA concentration of 400 mM.

Each mouse received 8 μg of plasmid DNA in a volume of 50 μl. The vector/EGTA mixture was administered to the mouse lung as described in Example 9 for rats, by intra-tracheal instillation.

One, three and seven days following the intra-tracheal instillation the mice were killed by cervical dislocation and the lungs perfused via the inferior vena cava with heparinised PBS until free of blood. Cell lysis buffer (Promega, Southampton, England) was added to each lung (4 μl/mg). The tissue was then homogenised two times, on ice, for 30 seconds each time (Polytron PT10-35, Philip Harris, Nottingham, England). Lung homogenates were is centrifuged at 10,000×g at 4 C for 10 minutes, then 20 μl of the supernatant were added to 100 μl of luciferase assay buffer (Promega) and luminescence measured in a luminometer (TD-20/20; Steptech Instruments, Stevenage, England). All results were repeated in triplicate and the mean calculated.

The total protein concentration in the lysate was measured using a protein assay kit (Bio-Rad, Hemel Hempstead, Herts, England) and using bovine serum albumin as a standard. Luciferase activity was expressed in relative light units (RLU) for each sample of lung lysate minus the background and normalised per mg of protein. The results are shown in FIG. 11.

As shown in FIG. 11, in the presence of 100 mM EGTA, transfection was enhanced about four-fold. However, 400 mM proved lethal in many cases.

### Example 15

### Methods and Materials

Airway Epithelial Cells

Human airway epithelial cell lines, a normal cell line 1HAEo- and a CF cell line 2CFSMEo-, were used in this study. 2CFS Meo-cells are immortalised, SV-40 T antigen transformed cells obtained from Dr D. Gruenert, UCSF. The 1HAEo-cells retain the morphologic and functional characteristics of epithelial cells and have been used in many different studies, see (Boussat et al (2000), Cozens et al (1992a). The 2CFSMEo-cells are ΔF508 heterozygous submucosal epithelial cells, see (Canonico et al (1996), Cozens et al (1996). Both cell lines were maintained in a humidified atmosphere of 95% air and 5% CO₂ at 37° C. in Eagle's minimum essential medium (MEM) supplemented with 10% fetal bovine serum, 2 mM .L-glutamine, 100 U/ml penicillin, and 100 μg/ml streptomycin (Sigma, Poole, UK) in 75 cm² culture flasks. Cells were seeded on transwell inserts of 0.4 μm pore size and 12 mm diameter (Costar, Bucks, UK) at a density of 10⁵ cells/0.5 ml/insert and were left to grow until fully confluent in about 3 days. The apical medium was then removed and growth medium was added basolateraly to leave the cells growing at an air-liquid interface. Confluent cells were grown for further 10-14 days and were monitored with an ohmmeter (EVOM; World Precision Instruments, Stevenage, UK) until the transepithelial resistance was greater than 300 Ω·cm before transfection. For immunofluorescence studies, the cells were seeded on coverslips in 24-well plates at a density of 10⁵ cells/ml/well and were left to grow for about 10-14 days until fully confluent. The state of confluence and development of tight junctions were also determined by the immunofluorescent detection of occludin, the structural and functional component of tight junctions (detailed below). Sub-confluent, proliferating cells were prepared by growing the cells for less then 3 days in growth medium prior to transfection.

Plasmid DNA

Three different plasmid DNA controls were used in this study.

Plasmid pCI-Lux was prepared by subcloning a luciferase gene from pGL3 control (Life Technologies, Paisley, UK) into the eukaryotic expression vector pCI (Promega, Southampton, UK). Plasmid pCIK-LacZ, containing cytoplasm-located bacterial β-Galactosidase gene, was obtained from Dr. Steve Hyde (Oxford). The plasmid pEGFP was commercially available (CLONTECH Laboratories UK Ltd., Hampshire, UK). All of these genes were driven by CMV promoter. The plasmids were amplified in Escherichia coli DH5_{α}, lysed with alkaline and prepared using an endotoxin-free kit (Qiagen, Crawley, UK). The DNA was washed with 70% ethanol and dissolved in water. The concentration of DNA was spectrophotometrically determined by its absorbance at A₂₆₀ and the purity was assessed with the ratio of A₂₆₀/A₂₈₀.

In Vitro Transfection of the Cells

The transfection vector complexes called “LID complexes” and “LID vectors” hereafter, were prepared in a procedure similar to that described in Example 1. Briefly, stock solutions of the three components of the LID vector complex were prepared first. Plasmid DNA (component D) was diluted in cell culture medium OptiMEM (Life Technologies, Paisley, UK) at a concentration of 0.01 μg/μl. The oligolysine-peptide component I comprised the integrin-targeted peptide CRRETAWACG, which targets the integrin α5 receptor, with a spacer XSXGA (X: ε amino hexanoic acid), and the oligolysine [K]₁₆, giving the sequence [K]₁₆XSXGACRRETAWACG (Zinsser Analytic, Maidenhead, UK). The peptide component I was dissolved in Opti-MEM at 0.1 mg/ml. The lipid component L was Lipofectin, a commercially available equimolar mixture of the cationic lipid N-[1-(2,3-dioleyloxy)propyl]-n,n,n-trimethylammonium chloride (DOTMA) and the neutral lipid dioleoyl phosphotidylethanolamine (DOPE), (Life Technologies, Paisley, UK) and was used from purchase at 1 mg/ml. Complexes were made by gentle mixing of the three components: lipofectin (L), peptide (I) and DNA (D), in the optimised weight ratio of 0.75:1.94:1 (L:I:D) which corresponds to a +/=charge ratio of about 3:1. The complexes were then left 30 minutes at room temperature before being diluted to a final concentration of 1 μg of DNA in 0.3 ml of Opti-MEM in transwells, or in 0.5 ml for 24-well plate. The cells were incubated with the complexes for 4 hours at 37° C. followed further culture in growth medium for 24 to 48 hours before being harvested for the assay of transgene expression.

Enhancement of Transfection

To enhance the efficiency of transfection in confluent epithelial cells, several agents were tested including EGTA (Sigma, Poole, UK), trypsin/EDTA (Sigma, Poole, UK), water, and mouse anti human E-cadherin antibody (Serotec Ltd, Oxford, UK). It was carried out firstly by comparing the effects of pretreatment of the cells with EGTA, water and Trypsin/EDTA with EGTA formulated into LID vector without pretreatment. Confluents cells were pre-incubated for 20-30 minutes at 37° C. with 2 mM EGTA, 0.05 g of trypsin/litre-0.02 g/litre of EDTA, water, PBS or OptiMEM as control, respectively. Cells were then washed twice with OptiMEM and transfected with LID complexes or LID formulated with EGTA (detailed below) for 4 hours as described above. To test the effects of anti-E-cadherin on transfection, confluent cells were pre-incubated with the antibody at increased concentrations of 2, 10 and 50 μg/ml for 60 minutes at 4° C., followed by 4-hour transfection with LID complexes in the presence of anti-E-cadherin of same concentrations or mouse IgG as a control, respectively.

Most of the enhancement experiments were carried out using the EGTA-formulation protocol (LID+EGTA), in which, EGTA was added to the OptiMEM containing LID complexes, at a final concentration of 2 mM. OptiMEM contains 0.9 mM CaCl₂ which is of half the level of conventional mediums. EGTA was first dissolved in PBS (pH7.3) to make a stock solution of 200 mM. After preparing the LID complexes, EGTA stock solution was added to the complexes to achieve a final concentration of 2 mM. The transfection incubation was performed in the presence of EGTA but was replaced with complete growth medium after 4 hours. Effects of EGTA on cell junctions were monitored by measuring the transepithelial resistance which dropped as early as about 10 minutes after exposure to EGTA and recovered at about 16-18 hours after removal of EGTA (data not shown).

Luminometric Assay for Luciferase

Twenty-four hours after transfection with pCI-luciferase, is cells were washed with PBS, lysed by adding 100 μl of Reporter Lysis Buffer (Promega, Southampton, UK) and detached from the plate by manual scraping using micropipette tips. Cell-free lysates were prepared by centrifugation at 2000 rpm for 5 minutes at 4° C. The activity of luciferase was assayed with a luciferase assay kit (Promega, Southampton, UK) on a Lucy-1 plate-reading luminometer (Anthos, Salzburg, Austria). The protein concentration of each sample was determined with Protein Assay Reagent (BioRad Laboratories, Hertfordshire, UK) by measuring absorbance at 595 nm and the specific activity of luciferase was expressed as relative light unit per milligram protein (RLU/mg protein).

Transfection Efficiency by Flow Cytometry Analysis of Green Fluorescent Pprotein:

Cells transfected with pEGFP were washed twice with PBS, harvested by digesting with trypsin-EDTA and fixed in 4% paraformaldehyde. The cells were then analysed by fluorescence-activated cell sorting (FACS) with a FACS sorter EPICS XL (Beckman Coulter Inc, Buckinghamshire, UK). Fluorescence for EGFP was measured at wavelength of 525±20 nm. Ten thousand cells were examined for the percentage of EGFP-positive cells by determining the percentage of highly fluorescent cells and subtracting the fluorescence of the untransfected control cells.

Assay of Cell Proliferation

Cell proliferation was assessed by incorporating 5-Bromo-2′-deoxy-uridine (BrdU) into cellular DNA detected by immunofluorescent staining using a monoclonal antibody. A BrdU Labelling and Detection Kit (Roche, Basel, Switzerland) was used, which contains the BrdU labelling reagent and the monoclonal mouse anti-BrdU. While the confluent cells were transfected with EGTA-formulated LID containing pEGFP for analysis of transfection efficiency, a control group was transfected with the same protocol but with 10 μM BrdU in the transfection medium for analysis of cell proliferation. Cells were maintained in the presence of BrdU throughout the 4 hour transfection incubation and for the following 24 hours incubation to label all cells that replicate during the transfection period. The BrdU-treated cells were then washed with PBS, harvested with trypsin/EDTA and fixed in 70% ethanol in 50 mM glycine buffer, pH 2.0, for 30 minutes at −20° C. The primary antibody, mouse anti-BrdU diluted 1:10 with incubation buffer provided in the kit, was added to the cells for a 60-minute incubation at 37° C. Cells were washed with PBS and incubated with a second layer antibody, Texas Red labelled horse anti-mouse IgG (Vector Laboratories, Peterborough, UK), for 30 minutes at 37° C. and then analysed by flow cytometry at the wavelength of 575±20 nm as detailed above. In some experiments, aphidicolin, a cell-proliferation inhibitor blocking the cell cycle in S-phase, was added to the cells to observe the effects on transfection efficiency. Aphidicolin was added to the growth medium (10 μg/ml) for 24 hours before transfection and maintained throughout the 4-hour transfection and the following 24 h period of incubation until the cells were harvested.

Binding of LID Vector to EGTA Treated Cells.

The effects of EGTA on cell binding were determined by transfection with LID complexes prepared with fluorescein (FITC) labelled peptide. Peptide-12 was labelled with FITC from a labelling kit (Calbiochem, San Diego, USA) and purified by dialyzing in PBS overnight, according to the manufacturer's instruction. The ability of FITC-conjugated peptide-12 to form complexes with plasmid DNA was determined by retardation test, in which the electrophoresis migration of DNA is retarded by forming complexes with the cationic peptide as described previously (Hart et al., 1995). The confluent cells in transwells were first incubated with 2 mM EGTA in Opti-MEM for 30 minutes at 37° C. to disrupt tight junctions and then cooled for 20 minutes at 4° C. The LID complexes prepared with FITC-labelled peptide-12 were also cooled to 4° C. for binding assay. The cells were then transfected with apically added LID-FITC for 60 minutes at 4° C., at which the complexes would only bind to cell surface and no internalisation would occur, see(Chu et al (1999), Cornelissen et al (1997), Im et al (1986). The surface binding of the LID complexes was further differentiated from the internalised LID by incubating the cells with 1 μg/ml crystal violet (Sigma, Poole, UK) for 10 minutes at 4° C., to quench the fluorescence of surface-bound LID complexes, see (Ma et al (1997), Van Amers & Van Strijp (1994). The mean numbers of fluorescent particles were then counted in 10 random microscope fields and comparison was made between the data obtained from EGTA-treated or untreated cells.

Immunofluorescence of BrdU, β-galactosidase and Occludin

To observe the double fluorescence labelling, the β-galactosidase-transfected and BrdU-labelled (as detailed above in Assay of cell proliferation) cells were fixed with 70% ethanol in 50 mM glycine buffer, pH 2.0, for 30 minutes at −20° C. The cells were then incubated with a mixture of two primary antibodies, the rabbit anti-p-galactosidase at 1:200 (Chemicon International Inc, Harrow, UK). and the mouse anti-BrdU at 1:10 (BrdU Labelling and Detection Kit; Roche, Basel, Switzerland), followed by incubation with a mixture of FITC-conjugated goat anti-rabbit IgG and texas red labelled horse anti-mouse IgG (Vector Laboratories, Peterborough, UK). Transwell inserts were then excised from the plastic holder and mounted on glass slides with aqueous mountant Vectashield (Vector Laboratories, Peterborough, UK). To observe the effects of EGTA on tight junction, the EGTA-treated cells were immunostained with a rabbit polyclonal antibody against tight junction protein occludin (Zymed, San Francisco, USA) diluted 1:10 with PBS. The antibody is specific to the C-terminal 150 amino acids of human occludin and has been used in many other studies, see (Jou et al (1998), Martin-Padura et al (1998). The confluent cells on transwells were incubated with 2 mM EGTA in Opti-MEM for 30 minutes at 37° C. and fixed in methanol for 20 minutes at 4° C. Washed with PBS, the cells were immunostained with anti-occludin for 60 minutes, followed by incubation with a fluorescein labelled goat anti-rabbit IgG (Vector Laboratories, Peterborough, UK) for 30 minutes at 37° C. The inserts were then excised and mounted on slides with Vectashield. Images were observed and captured with an inverted fluorescence microscope (Olympus IX70, Olympus Optical Company, London, UK) for transwell and a confocal microscope (Leica TCS SP, Exton, Pa., USA) for excised inserts.

All data shown are representative of at least 3 independent experiments and triplicate cell cultures were used for luminometry and flow cytometry analysis.

### Results

Comparison of Different Agents to Increase Transfection Efficiency

All transfections of the airway epithelial cells were performed in parallel in both 1HAEo- and 2CFSMEo-cells and similar results were observed in both cell types in most of the experiments. In preliminary studies of transfection of sub-confluent cells with LID vector, GFP transgene expression was observed in about 45% 1HAEo-cells (FIG. 11A) and 56% 2CFSMEo-cells (FIG. 11B) as determined by FACS analysis. However, fully confluent cells were much more resistant to transfection since the percentage of transgene positive cells fell to about 7% in both cell types (FIG. 11C and FIG. 11D.

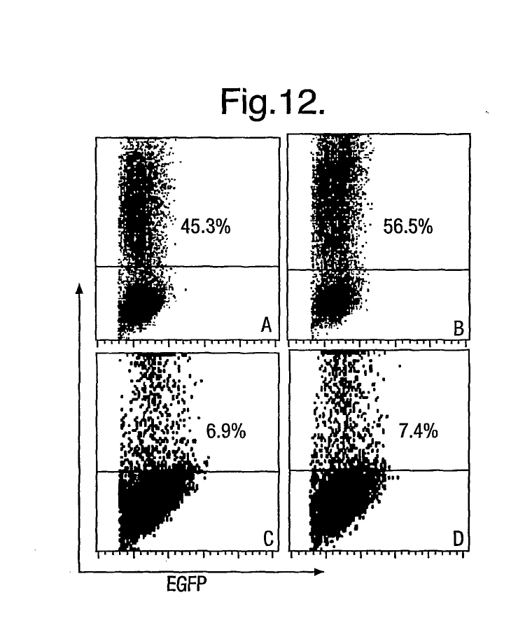

The poor transfection efficiency of confluent cells could be due to 1) the lack of availability of receptors on basolateral surfaces, or 2) the low rate of cell proliferation and the inability of plasmid DNA to enter the nucleus across the nuclear envelop. The first hypothesis was tested by treating the confluent epithelial cells with two agents known to disrupt cell junctions, EGTA and anti-cadherin monoclonal IgG, to augment gene transfer as reported previously for viral vectors, see Wang et al J Virol (1998); 72: 9818-9826 & Walters et al (1999). In the first protocol, confluent cells were incubated with 2 mM EGTA prior to transfection followed by 2 washes with Opti-MEM then transfected with the LID vector as usual. In the second protocol, LID complexes were formulated with 2 mM EGTA in the same solution (LID+EGTA) so that both vector and EGTA were added to cells simultaneously. In luciferase assays of transfected cells the EGTA pre-treatment protocol produced no enhancement of transfection while LID+EGTA treatment showed an eight-fold increase in luciferase transgene expression over the control transfection in the complete absence of EGTA (FIG. 12A). However, when the LID+EGTA formulation was used to transfect sub-confluent cells, there was no difference in transgene luciferase activities compared to control cells transfected without EGTA (FIG. 12B). In a third experiment cells were treated with anti-E-cadherin both prior and during transfection. A significant increase in transgene expression was observed in confluent cells transfected in the presence of anti-E-cadherin in a concentration-dependent manner compared to the transfection without anti-E-cadherin or with the presence of mouse IgG control (FIG. 12C). No significant difference was found in sub-confluent cells treated in the same way (FIG. 12D).

Therefore, two agents, EGTA and anti-E-cadherin were shown to enhance LID-mediated gene transfer to confluent epithelial cells while neither affected the transfection efficiency of sub-confluent cells. Since only confluent cells were affected, and both agents are known to affect the integrity of tight junctions, these results supported the hypothesis that transfection efficiency was reduced in confluent cells due to the restriction of receptors to the basolateral surfaces reducing binding and subsequent vector uptake. However, this did not eliminate the possibility that EGTA treatment may affect the transfection efficiency by increasing rate of cell division. The rate of mitosis is a major factor affecting the transfection efficiency of most plasmid-mediated vector systems.

Effects of EGTA on Transfection and Cell Proliferation

To test the hypothesis that EGTA treatment may enhance transfection by promoting cellular proliferation transfections were performed in confluent and subconfluent 1HAEo-cells with the LID+EGTA vector formulation. Transfection efficiency was determined from the percentage of cells transfected with the GFP reporter gene while the rate of proliferation was determined by immunodetection of BrdU incorporation by flow cytometry. Experiments were performed separately since a mutually compatible fixative for fluorescence analysis by flow cytometry could not be found.

Cells were transfected with pEGFP (A) or pEGFP plus EGTA(B), or pEGFP plus EGTA and aphidicolin (C). In parallel with the transfection, cells were labelled with BrdU (D) or BrdU plus ECTA (E), or BrdU plus EGTA and aphidicolin (F).

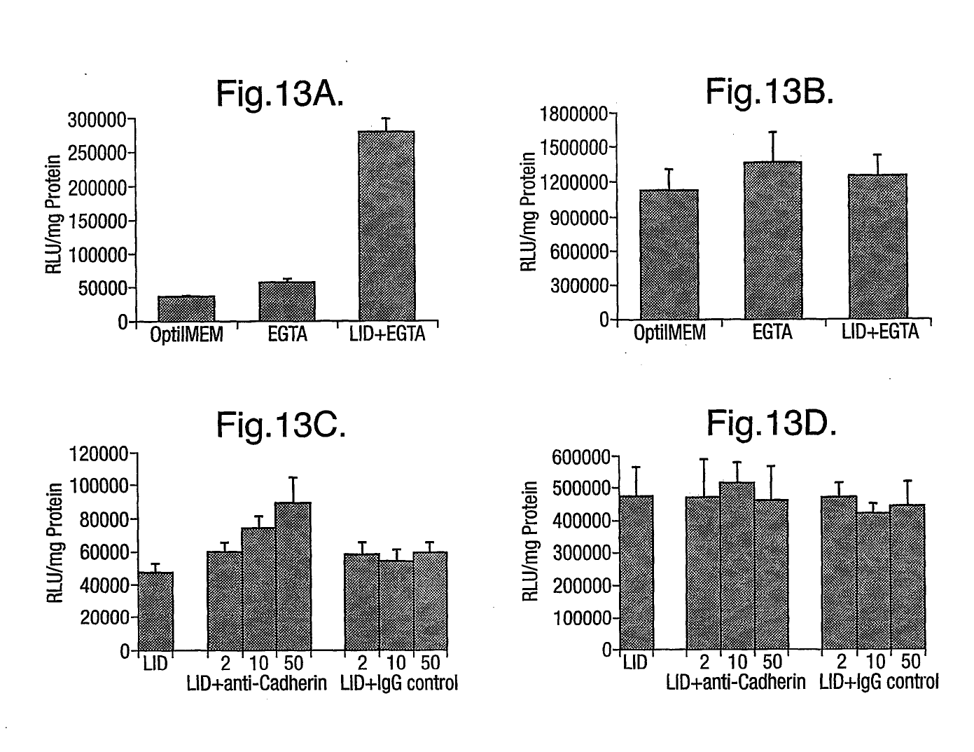

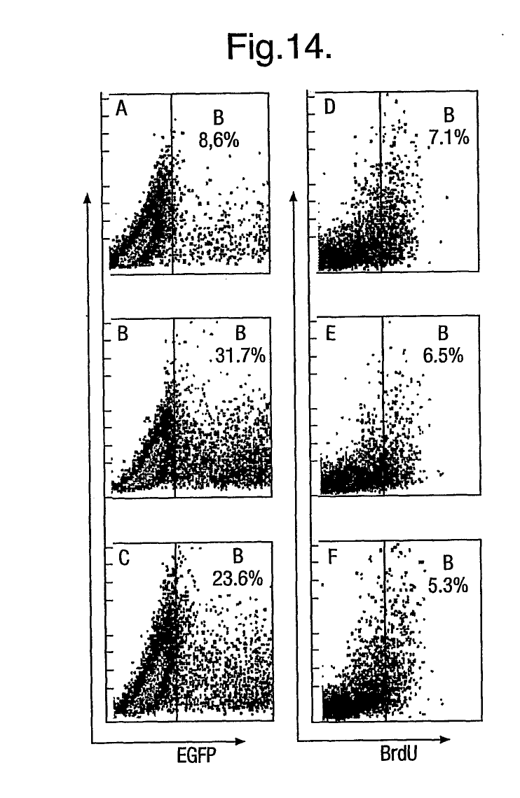

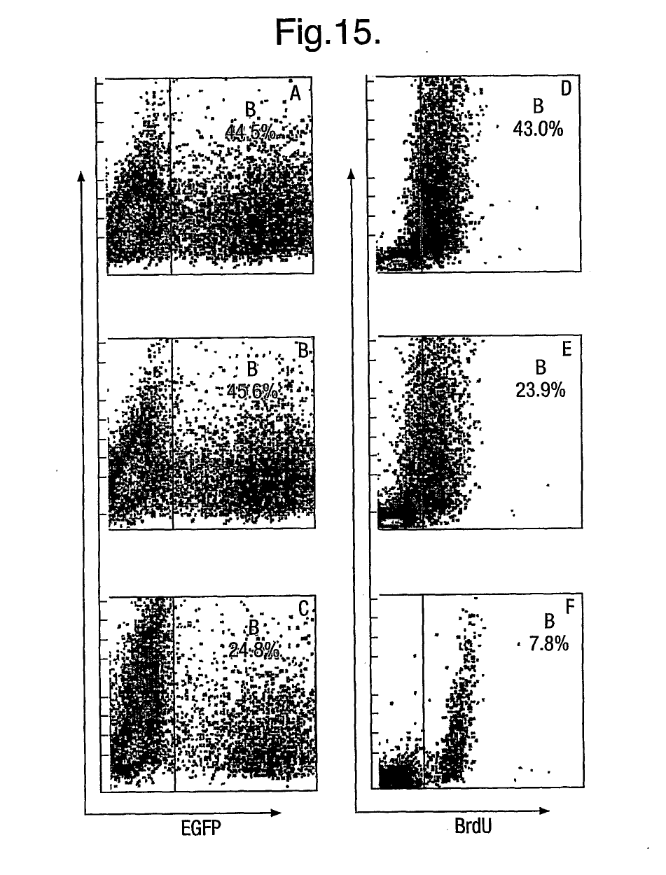

Confluent cells were transfected poorly by LID in the absence of EGTA, with only about 8% cells expressing the GFP transgene (FIG. 13A). Transfection of confluent cells with the LID+EGTA formulation, however, increased the transfection efficiency of confluent cells almost four-fold to approximately 31% (FIG. 13B). However, the rate of cell proliferation, as evident by the BrdU labelling, was not affected by EGTA treatment, remaining unchanged at about 6˜7% before (FIG. 13D) and after (FIG. 13E) EGTA treatment. In contrast, LID+EGTA transfection of subconfluent cells did not increase the transfection efficiency, with the transgene-positive cells being about 44-45% in both EGTA negative (FIG. 14A) and EGTA positive (FIG. 14B) transfections. EGTA treatment reduced the proliferation of subconfluent cells from about 43% in EGTA negative group (FIG. 14D) to about 24% in the EGTA positive group although this did not affect the transfection rate (FIG. 14E).

These results indicate that the formulation of the LID vector with 2 mM EGTA greately enhances transfection of confluent epithelial cells and that this enhacnement does not involve increased cellular proliferation. A possible explanation for these observations is that EGTA treatement enhacnes LID binding and subsequent uptake by exposing receptors on basolateral surfaces and that the LID vector is then able to transfect non-dividing cells.

LID+EGTA Transfection of Non-dividing Cells

Experiments were performed to test the hypothesis that the LID vector transfects non-mitotic cells by transfecting 1HAEo- in the presence of BrdU and aphidicolin, a cell cycle inhibitor, see (Jiang et al Hum. Gene Ther. 1998; 9:1531-5 1542.). EGTA enhancement of transfection was observed even in the presence of aphidicolin although the rate of cell proliferation indciated by BrdU incorporation was only 5% in confluent cells (FIG. 13F) and 7% in subconfluent cells (FIG. 14F). The LID+EGTA transfection rate of both confluent and subconfluent cells treated with aphidicolin was almost identical at 23% for confluent cells (FIG. 13C) and 24% for confluent cells (FIG. 14C). These results support the transfection of confluent cells and indicate that the LID vector transfects non-dividing cells with an efficiency at least four-fold higher than the rate of cell division. This data is consistent with the hypothesis that the LID vector transfects non-dividing cells.

To further test this proposal, cells were transfected with LID+EGTA containing a plasmid encoding the β-galactosidase reporter gene in the presence of BrdU then cells were anmaulysed by immunofluorescence mciroscopy to detect cells labelled with BrdU and expressing the reporter gene. Double immunofluorescence images of subconfluent cells transfected with LID+EGTA indicated a high level of proliferation. Many of the cells expressing the β-galactosidase reporter gene were also positive for BrdU indicating preferential transfection of dividing cells. Such double stained cells can be seen in FIG. 15A, one example is marked “g/r”. However amongst the confluent cells bot proliferation frequency and transfection efficiency were much lower (FIG. 15B). EGTA treatment of confluent cells however, markedly increased the proportion of β-galactosidase-positive cells (irregularly shaped stained cells in FIG. 15C, one example is marked “red”), while the proliferation rate as evident by BrdU labelling, remained low. Few cells were stained red, see rounded stained cells in FIG. 15C. While many transgene-positive subconfluent cells were proliferating cells as evident by the double labelling, most of the transgene expressing confluent cells were non-proliferative as they were not labelled by BrdU (FIG. 15A and FIG. 15C).

LID-Binding to EGTA-treated Confluent Cells

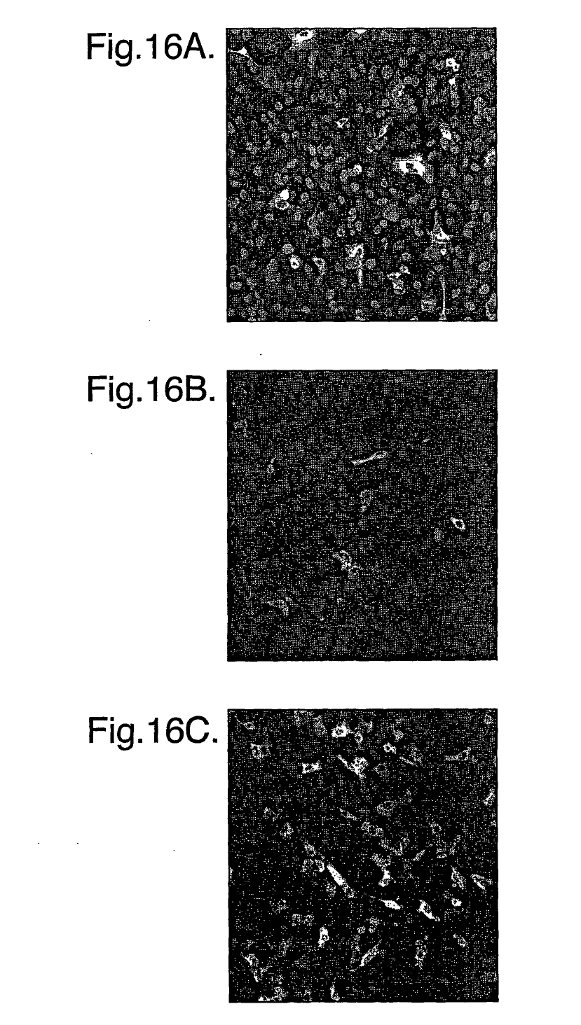

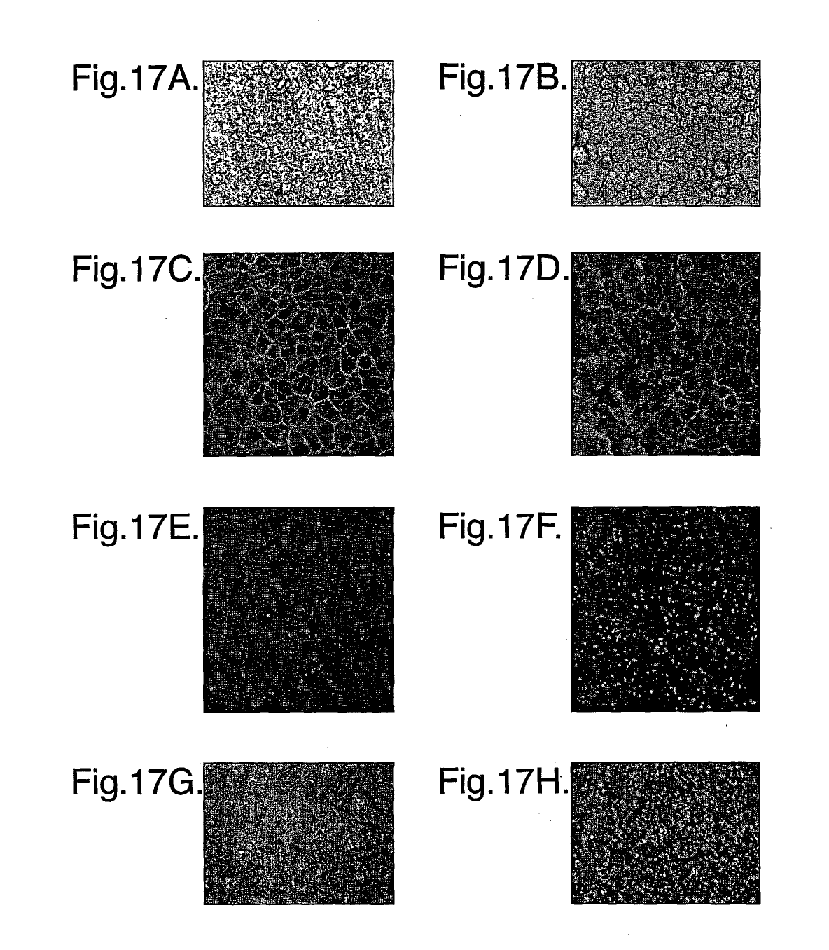

Since EGTA does not enhance transfection by increased cellular proliferation the enhancement of LID transfection may involve increased cell binding of the vector to exposed receptors on the basolateral surfaces. This proposal was tested by investigating the binding of fluorescently-labelled LID complexes prepared with FITC-conjugated peptide. Incubation of FITC-LID complexes with cells at 4° C. inhibited cellular internalisation by endocytosis and was confirmed by the observation that most of the cell associated fluorescence signal was quenched by crystal violet treatment. The crystal violet quenching method allows differentiation between internalised and surface-associated fluorescent material. A marked difference in LID complex binding efficiency was observed between the confluent cells transfected with and without EGTA. Only a few scattered dots representing FITC-labelled LID complexes bound to the cells without EGTA treatment (FIG. 16E) while in the presence of EGTA, binding of LID particles was greatly increased (FIG. 16F).

Exposure of receptors on basolateral surfaces of confluent cells may permit enhanced binding of the LID vector. This would require breakdown of the cellular tight junctions by 2 mM EGTA in OptiMEM. FIG. 16 shows photomicrographs of confluent 1HAEo-cells showing the effects of EGTA treatment. The images in left-hand column (A, C, E, G) shows the cells before EGTA treatment and the right-hand column (B, D, F, H) are the cells after EGTA treatment. They are representative images of the typical morphology of live confluent cells (A, B: ×400), conventional inverted microscope), immunofluorescence for tight junction protein occuludin (c, D: ×400 projected images, cofocal microscope), binding of LID complexes to cell surfaces (E, F: ×100, inverted confocal microscope) and the expression of transgene pEGFP (G, H: ×100, conventional inverted microscope). Confluent cells grown on transwell inserts until an impermeable cell sheet was formed (FIG. 16A). Tight junctions were clearly confined between cells as shown by positive immunofluorescent staining for occludin (FIG. 16C). Under these conditions few LID particles were bound to the cell surface (FIG. 16E) and only a small number of cells expressed transgene EGFP (FIG. 15G). After treatment with EGTA, however, the confluent cells rounded up, leaving spaces between each other (FIG. 16B). EGTA disrupted the tight junctions as evident by the broken lines of immunofluorescence for occludin (FIG. 16D). Now, many more particles of LID complexes to bind to the cell surfaces (FIG. 16F) and subsequently, many more cells were transfected with EGFP (FIG. 16H).

### Discussion

Many factors may affect the efficiency of gene transfer to airway epithelial cells, but the cell polarity and rate of proliferation are two of the main limiting factors. Modification of cell junctions and induction of cell proliferation has therefore been investigated with both viral and non-viral vectors (Bals et al (1999). These results demonstrated marked enhancement of gene transfer to confluent airway epithelial cells with the nonviral LID vector after disrupting the integrity of cell-junctions with EGTA, and increasing vector binding. EGTA Enhanced transfection was associated with the consequent increase in binding and enhancement of transfection was observed. More important, while a 4-fold increase in transfection efficiency was achieved the rate of cell proliferation as shown by BrdU-labelling, remained unaffected at about 7%. In addition, when transfections were performed in the presence of aphidicolin, in confluent and subconfluent cells, transfection with the LID vector formulated with EGTA was observed in at least 23% cells in both cases which is about 3-fold higher than the transfection without EGTA. Together with the evidence obtained by double immunofluorescence that most of transgene-positive confluent cells were non-proliferative, the present results show that LID vector transfects non-proliferating airway epithelial cells with a relative high efficiency.

Confluent airway epithelial cells are almost entirely mitotically quiescent. As evident in this Example, the proliferating BrdU-labelled cells accounted for about 43% of subconfluent but only 7% in confluent cells. Reduced cell proliferation would result in a low efficiency of transfection as reported by Fasbender that cells in mitosis (BrdU positive) were much more likely to express transgene than BrdU-negative cells, see (Fasbender et al (1997) and, by Wilke that growth-arrested cells were less efficiently transfected, see (Wilke et al (1996). The present study also showed the transfection was much more inefficient in confluent cells than in sub-confluent cells (8% vs. 44%). Increasing the rate of cell proliferation, therefore, would enhance gene transfer to airway epithelial cells as observed by transfecting the freshly seeded cells (Fasbender et al (1997) or stimulating cell proliferation with a growth factor (Wang et al (1998).

However, promotion of cell proliferation is not sufficient for efficient gene transfer to airway epithelial cells using a viral vector, see (Wang et al (1998). Wang and co-workers reported that although the rate of division of airway epithelial cells was increased to about 50% by stimulating with keratinocyte growth factor, none of the cells were transfected when the viral vector was applied to the apical membrane and gene transfer was observed only when the DNA was applied to the basal surface (16). Therefore, it would be critical to access the basolateral receptors for most of the current vectors to obtain efficient gene transfer. EGTA was found to be highly effective for promoting access of the LID vector to basolateral receptors, enhancing transfection efficiency with low toxicity, see (Wang et al (1998), Bals et al (1999), Chu et al (1999). In this Example, we have transfected confluent, non-proliferating airway epithelial cells with a relatively high efficiency by formulating the LID vector with EGTA and, by applying the LID vector to the apical surface.

Consistently, it has been reported that the low rate of cell proliferation limits gene transfer to airway epithelia by cationic lipid vector, see (Wilkie et al (1996). However, the majority of cells expressing transgene were not proliferative although the BrdU-positive cells are more likely to be transfected, see (Fasbender et al (1997). Matsui also reported that BrdU-positive cells were scattered throughout cell cluster whereas the transfection with liposome-DNA happened more frequently at the edge of cluster where the cells were more proliferative, see (Matsui et al (1997). In contrast, the results obtained in this Example showed that most of the confluent cells transfected with EGTA-formulated LID vectors were non-proliferating cells although many transfected subconfluent cells were proliferating cells. Taken all together, it suggested that, with the understanding that gene transfer could be facilitated by the high rate of proliferation, the state of proliferation, on the other hand, may not be a necessary factor for certain non-viral vectors to transfect certain cell types. It has previously been reported that microinjection of DNA/polyethylenimine complexes into the cytoplasm of several cell lines resulted in a higher expression of transgene compared with same amount of naked DNA, see (Palard et al (1998). Injection of DNA/polylysine complexes into cytoplasm of human fibroblasts led to a higher percentage of expressing cells as compared to DNA alone, see (Zauner et al (1999). Moreover, Vitiello suggested that polylysine might have a nuclear targeting activity as he found the co-localization of DNA and polylysine in cell nuclei by confocal microscopy,see (Vitiello et al (1996). All these data suggested that some polycations may facilitate the nuclear uptake of DNA complexes in certain cell types and explained in part the fact that lots of non-proliferating cells were transfected by our LID vector. However, this needs more intensive investigation especially in vivo to gain the convinced conclusion.

In summary, the results presented in this Example confirm that the efficient gene transfer to non-proliferating human airway epithelial cells using the synthetic nonviral vector LID, with adjuvant of a calcium chelator EGTA which transiently disrupted tight junctions to allow increased binding of LID complexes. No induction of cells proliferation was observed by EGTA treatment and most of transgene-positive cells were non-proliferative. This shows the ability of LID vector to transfect non-proliferating cells and its use in gene therapy of human respiratory diseases.

### References

1. Wu G Y, Wu C H. Receptor-mediated in vitro gene transformation by a soluble DNA carrier system. J Biological Chemistry 1987;262(10):4429-4432.

2. Wagner E, Cotten M, Mechtler K, Kirlappos H, Birnstiel M L. DNA-binding transferrin conjugates as functional gene-delivery agents: Synthesis by linkage of polylysine or ethidium bromide to the transferrin carbohydrate moiety. Bioconjugate Chemistry 1991;2:226-231.

3. Cotten M, Lange. Transferrin-polycation-mediated introduction of DNA into human leukemic cells: Stimulation by agents that affect the survival of transfected DNA or modulate transferrin receptor levels. PNAS 1990;87:4033-4037.

4. Ferkol T, Perales J C, Eckman E, Kaetzel C S, Hanson R W, Davis P B. Gene transfer into the airway epithelium of animals by targeting the polymeric immunoglobulin receptor. J Clinical Investigation 1995;95:493-502.

5. Curiel D T, Agarwal S, Wagner E, Cotten M. Adenovirus enhancement of transferrin-polylysine-mediated gene delivery. PNAS 1991;88:8850-8854.

6. Fernandez M A, Muno-Fernandez M A, Fresno M. Involvement of β1 integrins in the binding and entry of Trypanosoma cruzi into human macrophages. European J of Immunology 1993;23:552-557.

7. Wickham T J, Filardo E J, Cheresh D A, Nemerow G R. Integrin ávβ5 selectively promotes adenovirus mediated cell membrane permeabilization. J Cell Biology 1994;127(1):257-264.

8. Bergelson J M, Shepley M P, Chan B M C, Hemler M E, Finberg R W. identification of the integrin VLA-2 as a receptor for echovirus 1. Science 1992;255:1718-1720.

9. Logan D, Abu-Ghazaleh R, Blakemore W, et al. Structure of a major immunogenic site on foot-and-mouth disease virus. Nature 1993;362:566-568.

10. Isberg R R. Discrimination between intracellular uptake and surface adhesion of bacterial pathogens. Science 1991;252:934-938.

11. Almeida E A C, Huovilla A-P J, Sutherland A E, et al. Mouse egg integrin á6β1 functions as a sperm receptor. Cell 1995;81:1095-1104.

12. Clements J M, Newham P, Shepherd M, et al. Identification of a key integrin-binding sequence in VCAM-1 homologous to the LDV active site in fibronectin. J Cell Science 1994;107:2127-2135.

13. Lu X, Deadman J J, Williams J A, Kakkar V V, Rahman S. Synthetic RGD peptides derived from the adhesive domains of snake-venom proteins: evaluation as inhibitors of platelet aggregation. Biochemistry J 1993;296:21-24.

14. Koivunen E, Wang B, Ruoslahti E. Phage libraries displaying cyclic peptides with different ring sizes: ligand is specificities of the RGD-directed integrins. Biol/Technology 1995;13:265-270.

15. Koivunen E, Gay D A, Ruoslahti E. Selection of peptides binding to the á5β1 integrin from phage display library. J Biological Chemistry 1993;268(27):20205-20210.

16. Koivunen E, Wang B, Ruoslahti E. Isolation of a highly specific ligand for the á5β1 integrin from a phage display library. J Cell Biology 1994;124(3):373-380.

17. O'Neil K T, Hoess R H, Jackson A, Ramachandran N S, Mousa A, DeGrado W F. Identification of novel peptide antagonists for GPIIb/IIIa from a conformationally constrained phage peptide library. Proteins 1992;14:509-515.

18. Healy J M, Murayama O, Maeda T, Yoshino K, Sekiguchi K, Kikuchi M. Peptide ligands for integrin alpha v beta 3 selected from random phage display libraries. Biochemistry 1995;34:3948-3955.

19. Pasqualani R, Koivunen E, Ruoslahti E. A peptide isolated from phage display libraries is a structural and functional mimic of an RGD-binding site on integrins. J Cell Biology 1995;130:1189-1196.

20. Hart S L, Knight A M, Harbottle R P, et al. Cell binding and internalization by filamentous phage displaying a cyclic Arg-Gly-Asp-containing peptide. J Biological Chemistry 1994;269:12468-12474.

21. Hart S L, Harbottle R P, Cooper R, Miller A, Williamson R, Coutelle C. Gene delivery and expression mediated by an integrin-binding peptide. Gen Therapy 1995;2:552-554.

22. Wolfert M A, Seymour L W. Atomic force microscopic analysis of the influence of the molecular weight of poly(L)lysine on the size of polyelectrolyte complexes formed with DNA. Gene Therapy 1996;3:269-273.

23. Hart S L, Collins L, Gustaffson K, Fabre J W. Integrin mediated transfection with peptides containing arginine-glycine-aspartic acid domains. In press 1997.

24. Farhood H, Sebina A, Huang L. The role of dioleyl phos-phatidylethanolamine (DOPE) in cationic liposome is mediated gene transfer. Biochem Biophys Acta 1995;1235:289-295.

25. Anderson R, MacDonald I, Corbett T, Hacking G, Lowdell M W and Prentice H G. Human Gene Therapy 1997;8:1125-1135.

26. Blank R S, Thompson M M and Owens G K. Journal of Cell Biology 1988;107:299.

27. Bettinger, T., Remy, J. S. and Erbacher, P. (1999) Size reduction of galactosylated PEI/DNA complexes improves lectin-mediated gene transfer into hepatocytes. Bioconjug Chem, 10, 558-61.

28. Brunner, S., Sauer, T., Carotta, S., Cotten, M., Saltik, M. and Wagner, E. (2000) Cell cycle dependence of gene transfer by lipoplex, polyplex and recombinant adenovirus. Gene Ther, 7, 401-7.

29. Feero, W. G., Li, S., Rosenblatt, J. D., Sirianni, N., Morgan, J. E., Partridge, T. A., Huang, L. and Hoffman, E. P. (1997) Selection and use of ligands for receptor-mediated gene delivery to myogenic cells. Gene Ther, 4, 664-74.

30. Aberland, A., Knaus, T., Zaitsev, S. V., Stahn, R., Mistry, A. R., Coutelle, C., Haller, H. and Bottger, M. (1999) Calcium ions as efficient cofactor of polycation-mediated gene transfer. Biochim Biophys Acta, 1445, 21-30.

31. Han, J., Lim, M. and Yeom, Y. I. (1999) Receptor-mediated gene transfer to cells of hepatic origin by galactosylated albumin-polylysine complexes. Biol Pharm Bull, 22, 836-40.

32. Park, J. M., Yang, X., Park, J. J., Press, O. W. and Press, M. F. (1999) Assessment of novel anti-pl85HER-2 monoclonal antibodies for internalization-dependent therapies. Hybridoma, 18, 487-95.

33. Phillips, S. C. (1995) Receptor-mediated DNA delivery approaches to human gene therapy. Biologicals, 23, 13-6.

34. Reddy, J. A., Dean, D., Kennedy, M. D. and Low, P. S. (1999) Optimization of folate-conjugated liposomal vectors for folate receptor-mediated gene therapy. J Pharm Sci, 88, 1112-8.

35. Reddy, J. A. and Low, P. S. (2000) Enhanced folate receptor mediated gene therapy using a novel pH-sensitive lipid formulation. J Controlled Release, 64, 27-37.

36. Tseng, W. C., Haselton, F. R. and Giorgio, T. D. (1999) Mitosis enhances transgene expression of plasmid delivered by cationic liposomes. Biochim Biophys Acta, 1445, 53-64.

38. Uherek, C., Fominaya, J. and Wels, W. (1998) A modular DNA carrier protein based on the structure of diphtheria toxin mediates target cell-specific gene delivery. J Biol Chem, 273, 8835-41.

39. Wang, G., Davidson, B. L., Melchert, P., Slepushkin, V. A., van Es, H. H., Bodner, M., Jolly, D. J. and McCray, P. B., Jr. (1998) Influence of cell polarity on retrovirus-mediated gene transfer to differentiated human airway epithelia. J Virol, 72, 9818-26.

40. Wang, G., Zabner, J., Deering, C., Launspach, J., Shao, J., Bodner, M., Jolly, D. J., Davidson, B. L. and McCray, P. (2000) Increasing epithelial junction permeability enhances gene transfer to airway epithelia In vivo. Am J Respir Cell Mol Biol, 22, 129-38.

41. Wilke, M., Fortunati, E., van den Broek, M., Hoogeveen, A. T. and Scholte, B. J. (1996) Efficacy of a peptide-based gene delivery system depends on mitotic activity. Gene Ther, 3, 1133-42.

42. Wu, G. Y., Wilson, J. M., Shalaby, F., Grossman, M., Shafritz, D. A. and Wu, C. H. (1991) Receptor-mediated gene delivery in vivo. Partial correction of genetic analbuminemia in Nagase rats. J Biol Chem, 266, 14338-42.

43. Yano, L., Shimura, M., Taniguchi, M., Hayashi, Y., Suzuki, T., Hatake, K., Takaku, F. and Ishizaka, Y. (2000) Improved gene transfer to neuroblastoma cells by a monoclonal antibody targeting RET, a receptor tyrosine kinase [In Process Citation]. Hum Gene Ther, 11, 995-1004.

44. Zhang, F., Andreassen, P., Fender, P., Geissler, E., Hernandez, J. F. and Chroboczek, J. (1999) A transfecting peptide derived from adenovirus fiber protein. Gene Ther, 6, 171-81.

45. Fasbender, A., Zabner, J., Zeiher, B. G., and Welsh, M. J. (1997). A low rate of cell proliferation and reduced DNA uptake limit cationic lipid-mediated gene transfer to primary cultures of ciliated human airway epithelia. Gene Ther. 4: 1173-1180.

46. Matsui, H., Johnson, L. G., Randell, S. H., and Boucher, R. C. (1997). Loss of binding and entry of liposome-DNA complexes decreases transfection efficiency in differentiated airway epithelial cells. J.Biol.Chem. 272: 1117-1126.

47. Bals, R., Xiao, W., Sang, N., Weiner, D. J., Meegalla, R. L., and Wilson, J. M. (1999). Transduction of well-differentiated airway epithelium by recombinant adeno-associated virus is limited by vector entry. J.Virol. 73: 6085-6088.

48. Walters, R. W., Grunst, T., Bergelson, J. M., Finberg, R. W., Welsh, M. J., and Zabner, J. (1999). Basolateral localization of fiber receptors limits adenovirus infection from the apical surface of airway epithelia. J.Biol.Chem. 274: 10219-10226.

49. Anderson, J. M. and Van Itallie, C. M. (1995). Tight junctions and the molecular basis for regulation of paracellular permeability. Am. J. Physiol. 269: G467-G475.

50. Mitic, L. L. and Anderson, J. M. (1998). Molecular architecture of tight junctions. Annu.Rev.Physiol. 60: 121-142.

51. Takeichi, M. (1990). Cadherins: a molecular family important in selective cell-cell adhesion. Annu.Rev.Biochem. 59: 237-252.

52. Takeichi, M. (1991). Cadherin cell adhesion receptors as a morphogenetic regulator. Science. 251: 1451-1455.

53. Chu, Q., Tousignant, J. D., Fang, S., Jiang, C., Chen, L. H., Cheng, S. H., Scheule, R. K., and Eastman, S. J. (1999). Binding and uptake of cationic lipid:pDNA complexes by polarized airway epithelial cells. Hum.Gene Ther. 10: 25-36.

54. Boussat, S., Eddahibi, S., Coste, A., Fataccioli, V., Gouge, M., Housset, B., Adnot, S., and Maitre, B. (2000). Expression and regulation of vascular endothelial growth factor in human pulmonary epithelial cells. Am. J. Physiol Lung Cell Mol. Physiol. 279: L371-L378.

55. Cozens, A. L., Yezzi, M. J., Yamaya, M., Steiger, D., Wagner, J. A., Garber, S. S., Chin, L., Simon, E. M., Cutting, G. R., and Gardner, P. (1992a). A transformed human epithelial cell line that retains tight junctions post crisis. In Vitro Cell Dev.Biol. 28A: 735-744.

56. Canonico, A. E., Brigham, K. L., Carmichael, L. C., Plitman, J. D., King, G. A., Blackwell, T. R., and Christman, J. W. (1996). Plasmid-liposome transfer of the alpha 1 antitrypsin gene to cystic fibrosis bronchial epithelial cells prevents elastase-induced cell detachment and cytokine release. Am.J.Respir.Cell Mol.Biol. 14: 348-355.

57. Cozens, A. L., Yezzi, M. J., Chin, L., Simon, E. M., Finkbeiner, W. E., Wagner, J. A., and Gruenert, D. C. (1992b). Characterization of immortal cystic fibrosis tracheobronchial gland epithelial cells. Proc.Natl.Acad.Sci.U.S.A. 89: 5171-5175.

58. Cornelissen, C. N., Anderson, J. E., and Sparling, P. F. (1997). Energy-dependent changes in the gonococcal transferrin receptor. Mol.Microbiol. 26: 25-35.

59. Im, J. H., Frangakis, C. J., Rogers, W. J., Puckett, S. W., Bowdon, H. R., Rackley, C. E., Meezan, E., and Kim, H. D. (1986). Energy dependent insulin binding, internalization and degradation in isolated cardiac myocytes from normal and diabetic rats. J.Mol.Cell Cardiol. 18: 157-168.

60. Ma, J. A., Chapman, G. V., Chen, S. L., Penny, R., and Breit, S. N. (1987). Flow cytometry with crystal violet to detect intracytoplasmic fluorescence in viable human lymphocytes. Demonstration of antibody entering living cells. J.Immunol.Methods. 104: 195-200.

61. Van Amersfoort, E. S. and Van Strijp, J. A. (1994). Evaluation of a flow cytometric fluorescence quenching assay of phagocytosis of sensitized sheep erythrocytes by polymorphonuclear leukocytes. Cytometry. 17: 294-301.

62. Jou, T. S., Schneeberger, E. E., and Nelson, W. J. (1998). Structural and functional regulation of tight junctions by RhoA and Rac1 small GTPases. J.Cell Biol. 142: 101-115.

63. Martin-Padura, I., Lostaglio, S., Schneemann, M., Williams, L., Romano, M., Fruscella, P., Panzeri, C., Stoppacciaro, A., Ruco, L., Villa, A., Simmons, D., and Dejana, E. (1998). Junctional adhesion molecule, a novel member of the immunoglobulin superfamily that distributes at intercellular junctions and modulates monocyte transmigration. J.Cell Biol. 142: 117-127.

64. Pollard, H., Remy, J. S., Loussouarn, G., Demolombe, S., Behr, J. P., and Escande, D. (1998). Polyethylenimine but not cationic lipids promotes transgene delivery to the nucleus in mammalian cells. J.Biol.Chem. 273: 7507-7511.

65. Zauner, W., Brunner, S., Buschle, M., Ogris, M., and Wagner, E. (1999). Differential behaviour of lipid based and polycation based gene transfer systems in transfecting primary human fibroblasts: a potential role of polylysine in nuclear transport. Biochim.Biophys.Acta. 1428: 57-67.

66. Vitiello, L., Chonn, A., Wasserman, J. D., Duff, C., and Worton, R. G. (1996). Condensation of plasmid DNA with polylysine improves liposome-mediated gene transfer into established and primary muscle cells. Gene Ther. 3: 396-404.

1. A method of transfecting confluent cells or other slowly dividing or non-dividing cells that are in contact with each other, with a nucleic acid, which method comprises contacting the cells with a receptor-targeted vector comprising the nucleic acid and with an agent that disrupts cell-cell junctions under conditions suitable to effect transfection.

2. A method as claimed in claim 1, wherein the cells are epithelial or endothelial cells.

3. A method as claimed in claim 1 or claim 2, wherein the agent that is capable of disrupting cell-cell junctions is a calcium-binding or calcium chelating agent.

4. A method as claimed in claim 3, wherein the calcium binding or calcium chelating agent is EGTA.

5. A method as claimed in claim 4, wherein EGTA is used at a concentration of 1 mM or less, for example, from about 0.5 to 1 mM, especially about 1 mM in vitro or 25 to 200 mM, for example, 100 mM, in vivo.

6. A method as claimed in claim 1 or claim 2, wherein the agent that is capable of disrupting cell-cell junctions is an antibody directed to a substance involved in cell adhesion.

7. A method as claimed in claim 6, wherein the antibody directed to a substance involved in cell adhesion is an anti-cadherin.

8. A method as claimed in any one of claims 1 to 7, wherein the agent that disrupts cell-cell junctions is used at the same or substantially the same time as the vector.

9. A method as claimed in any one of claims 1 to 8, wherein the vector is a non-viral vector that is targeted to a cell-surface receptor.

10. A method as claimed in claim 9, wherein the vector is targeted to an insulin, asialoglycoprotein or transferrin receptor, or to a receptor on neuroblastoma cells, is folate conjugated to liposomes or is galactose for targeting liver cells

11. A method as claimed in claim 9, wherein the vector is targeted against an integrin receptor.

12. A method as claimed in claim 11, wherein the vector is a an integrin targeting transfection vector complex comprising (i) the nucleic acid, (ii) an integrin-binding component, especially an integrin-targeting peptide, (iii) a polycationic nucleic acid-binding component, especially an ologolysine, and (iv) a lipid component, especially, DOPE, DOTMA, DOSPA or a combination thereof.

13. A method as claimed in claim 12, wherein the polycationic nucleic acid-binding component is an ologolysine having from 10 to 20, especially 16, 17 or 18 lysine residues.

14. A method as claimed in claim 12 or claim 13, wherein the integrin-binding component is an integrin-binding peptide.

15. A method as claimed in any claim 14, where in peptide comprises the conserved amino acid sequence arginine-glycine-aspartic acid (RGD).

16. A method as claimed in any one of claims 12 to 15, wherein the integrin-binding peptide has at least two cysteine residues that form one or more double bond(s), thereby forming a cyclic peptide.

17. A complex as claimed in claim 13, wherein the integrin binding peptide consists of or comprises one of the following sequences: CRGDMFGC [SEQ.ID.NO.: 27]; CRGDMFGCG [SEQ.ID.NO.: 5]; CRGDMFGCA [SEQ.ID.NO.: 28]; CDCRGDCFCA [SEQ.ID.NO.: 29]; CRRETAWACA [SEQ.ID.NO.: 30]; CRRETAWAC [SEQ.ID.NO.: 13]; CRRETTAWAC [SEQ.ID.NO.: 31]; CRRETAWACG [SEQ.ID.NO.: 32]; CRGDMFGCGG [SEQ.ID.NO.: 33]; GPEILDVPST [SEQ.ID.NO.: 34]; CQIDSPCA [SEQ.ID.NO.: 35]; and CRRETAWACGKGACRRETAWACG [SEQ.ID.NO.: 36].

18. A method as claimed in any one of claims 13 to 17, wherein the integrin-binding peptide comprises a spacer element that is glycine-glycine (GG), glycine-alanine (GA) or is longer and/or more hydrophobic that the spacers GG and GA.

19. A method as claimed in claim 18 wherein the spacer element is XSXGA, in which S is serine, G is glycine, A is alanine and X is ε-amino hexanoic acid.

20. A process for expressing a nucleic acid in host cells, especially confluent cells or other slowly dividing or non-dividing cells that are in contact with each other, which comprises contacting the host cells in vitro or in vivo with a receptor-targeted vector comprising the nucleic acid and with an agent that disrupts cell-cell junctions under conditions suitable to effect transfection and then culturing the host cells under conditions that enable the cells to express the nucleic acid.

21. A process for the production of a protein in host Cells, especially confluent cells or other slowly dividing or non-dividing cells that are in contact with each other, which comprises contacting the host cells in vitro or in vivo with a receptor-targeted vector that comprises a nucleic acid that encodes the protein and with an agent that disrupts cell-cell junctions,under conditions suitable to effect transfection, culturing the host cells under conditions that enable the cells to express the protein, allowing the cells to express the protein, and obtaining the protein.

22. Confluent cells or other slowly dividing or non-dividing cells that are in contact with each other, transfected with a nucleic acid, and also the progeny of such cells.

23. A disease model for use in testing candidate pharmaceutical agents, which comprises confluent cells or other slowly dividing or non-dividing cells that are in contact with each other, transfected with a nucleic acid suitable for creating the disease model.

24. A pharmaceutical composition which comprises(i) a receptor targeted vector comprising a nucleic acid and (ii) an agent that disrupts cell-cell junctions, in admixture or conjunction with a pharmaceutically suitable carrier. The composition may be a vaccine.

25. A method for the treatment or prophylaxis of a condition caused in a human or in a non-human animal by a defect and/or a deficiency in a gene, which comprises administering to the human or to the non-human animal (i) a receptor-targeted vector comprising a nucleic acid suitable for correcting the defect or deficiency and (ii) an agent that disrupts cell-cell junctions.

26. A method for therapeutic or prophylactic immunisation of a human or of a non-human animal, which comprises administering to the human or to the non-human animal (i) a receptor-targeted vector comprising an appropriate nucleic acid and (ii) an agent that disrupts cell-cell junctions.

27. A method of anti-sense therapy of a human or of a non-human animal, comprising anti-sense DNA administering to the human or to the non-human animal (i) a receptor-targeted vector comprising the anti-sense nucleic acid and (ii) an agent that disrupts cell-cell junctions.

28. Use of (i) a receptor-targeted vector comprising a nucleic acid and (ii) an agent that disrupts cell-cell junctions for the manufacture of a medicament for the prophylaxis of a condition caused in a human or in a non-human animal by a defect and/or a deficiency in a gene, for therapeutic or prophylactic immunisation of a human or of a non-human animal, or for anti-sense therapy of a human or of a non-human animal.

29. A kit comprising (i) a receptor-targeted vector comprising a nucleic acid and (ii) an agent that disrupts cell-cell junctions.

30. A kit that comprises an agent that disrupts cell-cell junctions and the following items: (a) an integrin-binding component; (b) a polycationic nucleic acid-binding component, and (c) a lipid component. Such a kit may further comprise (d) either a nucleic acid or a plasmid or vector suitable for the expression of a nucleic acid, the plasmid or vector being either empty or comprising the nucleic acid.

31. A process, cell, disease model, pharmaceutical composition, method, use or kit as claimed in any one of claims 20 to 30, wherein the vector is as defined in any one of claims 12 to 19.
